(19) United States
(12) Patent Application Publication
(10) Pub. No.: US 2014/0303546 A1
(43) Pub. Date: Oct. 9, 2014

(54) LASER ASSISTED DELIVERY OF FUNCTIONAL CELLS, PEPTIDES AND NUCLEOTIDES

(71) Applicant: University of Miami, Miami, FL (US)

(72) Inventors: Evangelos V Badiavas, Miami, FL (US); Stephen C Davis, Miami, FL (US); Jill S Waibel, Miami, FL (US)

(21) Appl. No.: 14/128,344

(22) PCT Filed: Jun. 25, 2012
(86) PCT No.: PCT/US2012/043982

§ 371 Date: Jun. 13, 2014

Related U.S. Application Data
(60) Provisional application No. 61/500,946, filed on Jun. 24, 2011.

(51) Int. Cl. A61M 37/00

(52) U.S. Cl. CPC A61M 37/00; A61M 2037/0007
USPC 604/20

(57) ABSTRACT

The present invention relates to methods, uses, systems and kits for laser-assisted delivery of at least one bioactive agent to a subject. Specifically, laser light is used to create channel(s) in tissue of the subject, and the bioactive agent (s) is applied to the opening of the channel(s). The bioactive agent may be a cell, such as a functional cell, including stem cells, protein, peptide, peptide fragment, nucleic acid, nucleotide fragment, gene, pharmaceutical compound, therapeutic compound, medicament, small molecule, aptamer, or any combination of the above. Delivery may be accomplished locally or systemically, and can be used to treat local or systemic conditions or disorders. Functional reconstitution and gene therapy is also possible with the present invention. Other uses include directing bioactive agent migration, stimulating endogenous stem cell circulation, treating biofilms, and regrowth of hair. Tissue explants are also incorporated in some embodiments.

This application claims priority to International Patent Application Number PCT/US2012/043982, filed Jun. 25, 2012, which claims priority to U.S. provisional patent application Ser. No. 61/500,946, filed Jun. 24, 2011.

### FIELD OF THE INVENTION

This invention relates generally to the field of therapeutic delivery. More particularly, it concerns methods of cell, peptide and nucleotide delivery using laser technologies.

### BACKGROUND

The accurate and effective delivery of pharmaceutical drugs, therapeutic agents, gene therapy, and other biologically active compounds to the sites most in need thereof has been a prolonged and continuing problem in medicine. As advances in science and technology provide increasingly sophisticated solutions to disorders, such as stem cells to regenerate tissue, proteins or genes which are synthesized or produced by specifically-engineered cells to restore missing function, micelles and other molecular vehicles, and nanotechnology for transporting drugs or therapeutics, etc., delivering such compounds to a specific target site for treatment remains a limiting factor.

Injection as a delivery method of compounds such as drugs, therapeutics, small molecules, vaccines, etc. is commonly performed. However, such treatment is principally only delivered to tissue that can be accessed by a needle, or enters the bloodstream and circulates through the body in a non-specific manner, such that treatment may or may not reach the site where it is needed, and if it does reach the site at all, it may be in such small concentrations or amounts as to be ineffective or minimally effective.

For example, some known stem cell therapies require injection or administration of stem cells through an IV to deliver the stem cells to the subject or patient. Upon injection or IV introduction, the stem cells circulate throughout the body. Accordingly, a large number of stem cells, approximately 200 million cells or more, must be initially introduced to ensure some of them make it to their intended target. Other known methods of stem cell therapy require the introduction or application of hematopoietic hormones or growth hormones to stimulate the subject's body to release its own stem cells for therapy and repair, rather than introduce foreign stem cells. This method too, however, requires time for the body to produce and release more stem cells, and the hormones used to induce such production can have significant, possibly negative, side effects that may make it an unacceptable method of stem cell therapy.

Other delivery methods seek to guide compounds to target sites within the body. For example, compounds and compositions have also been engineered to include magnetic components, such that upon injection or entry into the body, a magnetic field may be applied to the body and adjusted to magnetically guide the compound to a specific site of need. At certain strengths and/or exposure levels, however, the magnetic field employed in this approach can have negative effects on the body of the subject, and may cause unintended damage. Further, the body must then process the magnetic component, which may itself cause damage to internal organs, tissues, or cells. As a result, while producing some increase in targeting, significant limitations remain.

As yet another mode of delivery, the delivery of DNA- and protein-based therapeutics, pharmaceutically active compounds, and even small molecules has been accomplished through the use of microneedles or transdermal patches. However, such delivery is limited to the skin and the therapeutics are generally only delivered to the precise location where they are placed. Moreover, micro needles deliver material by traditional puncture injury and administration under some degree of pressure or artificial gradient. If needles are placed temporarily, the wound created for delivery will collapse on itself and rapidly close, often expelling the material delivered and limiting the further entrance of any additional materials. If microneedles are left in place for a prolonged period, abnormal micro environmental changes will take place. Pseudoepithelializtion at the needle/tissue interface and chronic inflammatory changes will occur. These changes create a barrier to further administration of materials. The presence of a foreign body also increases the risk for infection. Systemic delivery by these methods is at best limited to very small molecules. Transdermal delivery of materials requires that the skin be modified in its barrier function by gross chemical or physical means. These methods can prove to be locally toxic or damaging to the skin. Disruption of the barrier function of skin is considered a primary or contributing factor a majority of skin disorders. Indiscriminate or gross barrier function alteration of barrier function over anything larger than a small patch would not be desirable from a therapeutic standpoint. Molecules amenable for systemic delivery by these methods would have to be limited by small size and specific chemical characteristics such as, charge, solubility and compatibility with the barrier function altering agents. In addition, transdermal delivery does not allow for penetration of therapeutics directly into deeper layers of the skin, e.g. dermal wounds.

Accordingly, what is needed is a more effective method of delivering functioning cells, proteins, nucleic acids, and other biologically active compounds, both locally, including over broad areas, and systemically throughout the body and to distant sites, to a subject. It would also be highly beneficial to have a way of delivering functional cells transdermally that remain viable and functional and can provide therapeutic and/or beneficial responses within the body, such as at specific sites.

Furthermore, it is recognized that laser technology is not in and of itself new, and has been used for many years, primarily in the area of dermatology. For example, ablative and non-ablative lasers are commonly used in cosmetic applications, such as in the reduction of wrinkles, stimulation of new collagen growth, and correction of sun-related damage. They are also commonly utilized in localized hair removal. Notably, however, known laser treatments focus on limiting the amount of laser light applied to the subject, and therefore, the amount of damage caused. Most known medical functions of lasers are based on the concept of selective photothermolysis, which states that laser light of a specific wavelength can destroy a target containing the adequate chromophore without damaging the surrounding tissue. (Anderson et al., 1993). The goal thus far of lasers has been to destroy specific tissues. For this reason, lasers have safety boundaries.

For instance, most known laser treatments require safety features on the laser, control of the laser intensity, and/or the application of laser light in bursts rather than continuous exposure. More recently, fractional ablative lasers have been developed that permit the application of laser light in discrete segments, similar to pixels on a screen, rather than a wide area in order to limit the amount of laser light applied and damage caused thereby. (Manstein D D, 2004). Moreover, known laser treatments have a superficial application, in that they are applied to the surface of the skin and produce minimal penetration into the epidermis and dermis, such as between 10 and 1000 microns depending on laser type. Usually, treatments do not go beyond 300 microns in depth for the majority of aesthetic treatments. As such, it has not been contemplated to utilize such lasers, at increased power levels, to create actual channels within a subject's tissue to create new tissue or organs.

### SUMMARY

The present invention use lasers in ways they have not been used previously, and for purposes for which they have not been contemplated. Specifically, the present invention is directed to a method of delivering bioactive agent(s) to a subject using a laser to define channels of a certain depth into the tissue of a subject for systemic therapy. In this regard, the present methods use a laser(s) at a more powerful intensity than they are used in current cosmetic applications, to create channels that are deeper than what is achieved by current laser treatments. Lasers are based on physics and precise control of light to tissue. Laser devices can be controlled to deliver light to any desired depth in any organ. The deeper penetration required, the tunable laser systems are able to change parameters to achieve the desired anatomical level.

Moreover, rather than utilizing the laser merely for the effect the laser itself will have on the surface tissue, the present method uses lasers to deliver functioning cells, genes, nucleotides, peptides, or other bioactive agent(s) to a site within the body, either locally or systemically—a purpose for which lasers have not been contemplated or utilized to date. Indeed, the laser ablation of tissue which creates the channel(s) initiates a biochemical and/or biophysical response, which the present invention utilizes to facilitate the movement and recruiting of applied bioactive agent(s) to the surrounding tissue and beyond. For instance, the bioactive agent(s) applied may further be able to access circulatory systems of the body, such as the hematopoietic or lymph system, in order to migrate or be recruited or transported to distant sites where they may be therapeutically active, and in a manner which may be more effectively tolerated and/or integrated by the body.

In at least one embodiment, the method of the present invention comprises applying a laser, such as a beam of laser light, to a target tissue at a local site, creating at least one channel in the tissue of the target local site, and administering the bioactive agent(s) to the laser-treated target local site at the channel(s). In some embodiments, the target tissue is the epidermis of skin. Accordingly, the present invention permits, for the first time, transdermal delivery of functional cells or other bioactive agent, in a more effective manner than other known methods such as injection. The laser treatment and channels created thereby provide a unique microenvironment for the bioactive agent that promotes the migration of the bioactive agent down into the channel(s) and into the surrounding tissue, as well as systemic migration therefrom, such as for treatment of disease, growth of new organs and tissue regeneration. Given the many possible uses of the present invention which are discussed in greater detail hereinafter, including functional reconstitution, treatment of systemic conditions that affect the entire body, or treating conditions systemically so that introduction of the bioactive agent occurs in a different location than the site affected by the condition and which the bioactive agent acts, the present invention is remarkable.

Although many embodiments create the laser-generated channels at the skin of the subject, some embodiments contemplate the target site is an organ or other internal tissue such as may be exposed during open or laparoscopic surgery. The target tissue may even comprise a wound, such as a burn, injury, or lesion.

In one preferred embodiment, the laser used within the present method is a fractional ablative laser, such that the laser light as applied to the skin or other tissue creates a channel therethrough. The location, diameter, depth, density, and other characteristics of the channel(s) can be precisely controlled and dictated by the particular settings of the laser. Indeed, in some preferred embodiments, a matrix of a plurality of channels is created using the laser.

Once the laser is applied and channels are created in the target tissue, the bioactive agent(s) is applied to the channel(s) at or near the surface or opening of the channel(s). The bioactive agent(s) may comprise viable and/or functioning cells, protein(s), peptide(s), peptide fragment(s), nucleic acid(s), nucleotide fragment(s), gene(s), pharmaceutical compound(s), therapeutic compound(s), medicament(s), small molecule(s), aptamer(s), and combinations thereof. In particular, the bioactive agent(s) are any compound, composition, or matter that is capable of rendering a biological affect when applied to the subject. Further, in some embodiments, the biological agent(s) may be transduced, engineered, or otherwise specifically designed to deliver particular proteins or secreted factors to the subject upon administration at the target site, and/or can be accompanied by other factors, such as carriers, solvents, adjuvents, etc. Once applied at the target site, because of the manner in which the laser creates the channels and in part due to the natural physiological and biochemical response that results from laser ablation, the subject's innate and endogenous mechanisms are triggered to draw the applied bioactive agent(s) down the channel(s) and into the local tissue surrounding the channel, and systemically to distal sites as well, for maximum effect.

As noted, in some embodiments, the method of the present invention is used to deliver bioactive agent(s) to a local site on a subject, such as at the same tissue being treated with the laser. In other embodiments, however, the method of the present invention may also be used to deliver bioactive agent(s) systemically to sites distant from the local site that is treated with the laser and to which the bioactive agent(s) is applied. For instance, application of the laser and bioactive agent(s) at a first site will render an effect at a different site, that distant site in some cases being a wound or injury, and in other situations it may be a distant site that is affected by a disorder, or lacks a gene or protein. This is due in part to the introduction of the bioactive agent(s) at site of increased endogenous activity that is responding to the laser ablation. The distal site may also optionally be subjected to laser application to create laser-ablated channel(s) at the distal site, so as to more efficiently direct the bioactive agent to such site. Indeed, in some embodiments the creation of laser-ablated channel(s) at a particular site on a subject may be used to direct circulating bioactive agent, such as cells including stem cells, to the site of laser application for beneficial affect there.

In still other embodiments of the present method, a laser is applied to a tissue explant, creating at least one channel therein, and at least one bioactive agent is applied to the tissue explant. The resulting seeded tissue explant is then implanted into a subject, and the bioactive agent(s) carried thereby is drawn from the explant tissue into the body of the subject.

As indicated above, the method of the present invention has a wide variety of applications. For example, the method can be used to treat locally, systemically, or through tissue explant. The method can also be used to treat wounds, such as burns, other injuries including acute injury, devitalized skin, and/or to treat skin disorders, such as but not limited to: genetic based skin disease, inflammatory skin disease, wound healing, skin and wound infections, scar reduction, tissue remodeling, skin regeneration, improved cosmesis of the skin, such as treatment of rhytids and solar elstosis, pigmentary disorders, and alopecia or hair loss, both medical and cosmetic, and revascularization. The method can also be used to treat systemic disease or disorders, such as but not limited to: treatment of distant organ damage such as myocardial infarction and chronic lung disease; reconstitution of immune function, hematopoietic function, or other lost function; treatment of vascular disorders such as peripheral artery disease, stroke, and lymphedema, and distant sites of injury. Yet another application of the present method can be in vaccination and immune tolerance approaches, and for functional reconstitution of cells, genes, or peptides. The present invention can also be used to stimulate increased circulation of the subject's own endogenous stem cells, through the application of laser light to create channel(s) and administration of a bioactive agent(s) there. The invention can also be used to grow and/or regrow hair.

In addition to the above methods and uses, the present invention is also directed to a system for delivering bioactive agent(s) to a subject, comprising a laser instrument capable of producing laser light and at least one bioactive agent. The bioactive agent(s) may comprise viable and/or functioning cells, protein(s), peptide(s), peptide fragment(s), nucleic acid(s), nucleotide fragment(s), gene(s), pharmaceutical compound(s), therapeutic compound(s), medicament(s), small molecule(s), aptamer(s), and combinations thereof. The system may be structured to deliver the bioactive agent(s) locally to a particular site, systemically throughout the body, and systemically to a distal site spaced apart from the target site of laser application. In still other embodiments, the system further comprises a tissue explant that contains the bioactive agent(s).

The present invention is also directed to kits of parts comprising bioactive agents, reagents, and instructions for use thereof. The kits may also include a laser and instructions for use of a laser in combination therewith. In other embodiments, the kits also include a tissue explant which may already be seeded with bioactive agent, or may include instructions for applying laser to the tissue explant and administration of the bioactive agent to create a seeded explant, as well as instructions for implantation thereof.

The methods, uses, systems, and kits described herein can be used in connection with pharmaceutical, medical, clinical, and veterinary applications, as well as fundamental scientific research and methodologies, as would be identifiable by a skilled person upon reading of the present disclosure. These and other objects, features and advantages of the present invention will become clearer when the drawings as well as the detailed description are taken into consideration.

In a first embodiment, the present invention provides a method of delivering one or more bioactive agents to a subject, comprising: applying laser light to a target site on the subject, wherein the target site is spaced apart from a distal site to be affected by at least one bioactive agent, creating at least one channel in the tissue of the target site with the laser light, and administering one or more bioactive agent to the target site at said channel for migration into the channel and to the distal site.

In a second embodiment, the present invention provides a method according to the first embodiment, wherein creating at least one channel with laser light causes a reaction in the subject that promotes migration of the one or more bioactive agent.

In a third embodiment, the present invention provides a method according to the first embodiment, wherein applying laser light and creating at least one channel are sufficient to create an injury response at least 250 microns below the surface of the tissue of the target site.

In a fourth embodiment, the present invention provides a method according to the third embodiment, wherein the injury response is a laser-generated injury response.

In a fifth embodiment, the present invention provides a method according to the first embodiment, wherein creating at least one channel includes creating at least one channel having a predetermined depth of at least 300 microns as measured from a surface of the tissue at the target site.

In a sixth embodiment, the present invention provides a method according to the fifth embodiment, wherein creating at least one channel includes creating at least one channel having a predetermined depth in the range of about 300 to 2000 microns as measured from the surface of the tissue at the target site.

In a seventh embodiment, the present invention provides a method according to the first embodiment, wherein the tissue at the target site is skin.

In an eighth embodiment, the present invention provides a method according to the seventh embodiment, wherein creating at least one channel includes creating at least one channel having a depth at least sufficient to penetrate the dermal layer of skin.

In a ninth embodiment, the present invention provides a method according to the first embodiment, wherein applying a laser includes applying ablative laser light to the target site.

In a tenth embodiment, the present invention provides a method according to the ninth embodiment, wherein applying a laser includes applying fractional ablative laser light to the target site.

In an eleventh embodiment, the present invention provides a method according to the first embodiment, wherein the at least one channel includes an opening at the surface of the tissue at the target site.

The method as recited in claim 11 wherein applying one or more bioactive agents includes applying one or more bioactive agents at the opening of the at least one channel.

In a thirteenth embodiment, the present invention provides a method according to the first embodiment, wherein the at least one channel is a coagulated channel.

In a fourteenth embodiment, the present invention provides a method according to the first embodiment, wherein the at least one channel comprises a width of about 400 microns or less.

In a fifteenth embodiment, the present invention provides a method according to the fourteenth embodiment, wherein the at least one channel comprises a width of about 200 microns or less.

In a sixteenth embodiment, the present invention provides a method according to the fifteenth embodiment, wherein the at least one channel comprises a width in the range of about 10 to 200 microns.

In a seventeenth embodiment, the method according to the first embodiment further comprises creating a plurality of channels of predetermined depths in the tissue at the target site.

In an eighteenth embodiment, the method according to the seventeenth embodiment further comprises creating a plurality of channels in the range of about 400 to 800 channels per centimeter square of target site.

In a nineteenth embodiment, the present invention provides a method according to the seventeenth embodiment, wherein each of the plurality of channels defines an opening in the tissue of the target site.

In a twentieth embodiment, the present invention provides a method according to the nineteenth embodiment, wherein applying one or more bioactive agents includes applying one or more bioactive agents at the openings of the plurality of channels.

In a twenty-first embodiment, the present invention provides a method according to the first embodiment, wherein the at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In a twenty-second embodiment, the present invention provides a method according to the first embodiment, wherein the one or more bioactive agents includes at least one type of functional cell.

In a twenty-third embodiment, the present invention provides a method according to the twenty-second embodiment, wherein administering includes administering approximately 1 million cells or less to the at least one channel at the target site.

In a twenty-fourth embodiment, the present invention provides a method according to the twenty-second embodiment, wherein the one or more bioactive agents includes at least one type of stem cell.

In a twenty-fifth embodiment, the present invention provides a method according to the twenty-fourth embodiment, wherein the one or more bioactive agents includes mesenchymal stem cells.

In a twenty-sixth embodiment, the present invention provides a method according to the twenty-second embodiment, wherein the one or more bioactive agents includes at least one type of adult cell.

In a twenty-seventh embodiment, the present invention provides a method according to the twenty-second embodiment, wherein the one or more bioactive agents includes at least lymphocytes.

In a twenty-eighth embodiment, the present invention provides a method according to the twenty-second embodiment, wherein the one or more bioactive agents includes at least one type of progenitor cell.

In a twenty-ninth embodiment, the present invention provides a method according to the twenty-second embodiment, wherein the one or more bioactive agents includes a mixture of functional cells.

In a thirtieth embodiment, the present invention provides a method according to the twenty-ninth embodiment, wherein the one or more bioactive agents includes a mixture of at least one type of stem cell and a second type of cell.

In a thirty-first embodiment, the present invention provides a method according to the thirtieth embodiment, wherein the second type of cell is progenitor cells.

In a thirty-second embodiment, the present invention provides a method according to the thirtieth embodiment, wherein the second type of cell is adult cells.

In a thirty-third embodiment, the present invention provides a method according to the thirtieth embodiment, wherein the stem cells are mesenchymal stem cells.

In a thirty-fourth embodiment, the present invention provides a method according to the thirtieth embodiment, wherein the at least one type of stem cell and the second type of cell have different potencies.

In a thirty-fifth embodiment, the method according to the first embodiment further comprises applying laser light to the distal site, creating at least one channel at the distal site so as to direct the at least one bioactive agent to the distal site.

In a thirty-sixth embodiment, the present invention provides a method according to the first embodiment, wherein the at least one bioactive agent includes at least one corrective gene or corrective gene product capable of providing therapeutic benefit for a condition resulting from a corresponding aberrant gene or aberrant gene product.

In a thirty-seventh embodiment, the present invention provides a method according to the thirty-sixth embodiment, wherein the corrective gene product comprises RNA or protein.

In a thirty-eighth embodiment, the present invention provides a method according to the thirty-sixth embodiment, wherein the at least one bioactive agent comprises cells having at least one corrective gene or corrective gene product and capable of expressing the corrective gene or corrective gene product.

In a thirty-ninth embodiment, the present invention provides a method according to the thirty-sixth embodiment, wherein the at least one bioactive agent comprises a mixture of a first set of cells having at least one corrective gene or corrective gene product and capable of expressing said corrective gene or corrective gene product, and a second set of cells defined as at least one type of stem cell.

In a fortieth embodiment, the present invention provides a method of delivering one or more bioactive agents to a subject, comprising: applying laser light to a target site on the subject, wherein the target site is spaced apart from a distal site to be affected by at least one bioactive agent, creating at least one channel of a predetermined depth in the tissue of the target site and defining an opening of the at least one channel at a surface of the target site tissue, administering one or more bioactive agents at the opening of the at least one channel for migration into the channel and to the distal site, and applying laser light to the distal site, thereby creating at least one channel at the distal site so as to direct the at least one bioactive agent to the distal site.

In a forty-first embodiment, the present invention provides a method according to the fortieth embodiment, wherein the predetermined depth of the at least one channel is at least 300 microns as measured from a surface of the tissue at the target site.

In a forty-second embodiment, the present invention provides a method according to the forty-first embodiment, wherein the predetermined depth of the at least one channel is in the range of about 300 to 2000 microns as measured from the surface of the tissue at the target site.

In a forty-third embodiment, the present invention provides a method according to the fortieth embodiment, wherein the tissue at the target site is skin.

In a forty-fourth embodiment, the present invention provides a method according to the forty-third embodiment, wherein creating at least one channel includes creating at least one channel having a depth at least sufficient to penetrate the dermal layer of skin.

In a forty-fifth embodiment, the present invention provides a method according to the fortieth embodiment, wherein the at least one channel comprises a width of about 400 microns or less.

In a forty-sixth embodiment, the present invention provides a method according to the forty-fifth embodiment, wherein the at least one channel comprises a width of about 200 microns or less.

In a forty-seventh embodiment, the present invention provides a method according to the forty-sixth embodiment, wherein the at least one channel comprises a width in the range of about 10 to 200 microns.

In a forty-eighth embodiment, the present invention provides a method according to the fortieth embodiment, wherein applying a laser includes applying ablative laser light to the target site.

In a forty-ninth embodiment, the present invention provides a method according to the forty-eighth embodiment, wherein the laser light is generated by a fractional ablative laser.

In a fiftieth embodiment, the present invention provides a method according to the fortieth embodiment, wherein the at least one channel at the target site is coagulated.

In a fifty-first embodiment, the present invention provides a method according to the fortieth embodiment, wherein the at least one channel at the distal site is coagulated.

In a fifty-second embodiment, the method according to the fortieth embodiment further comprises creating a plurality of channels in the tissue of the target site.

In a fifty-third embodiment, the method according to the fifty-second embodiment further comprises creating a plurality of channels in the range of about 400 to 800 channels per centimeter square of target site.

In a fifty-forth embodiment, the method according to the fortieth embodiment further comprises creating a plurality of channels in the tissue of the distal site.

In a fifty-fifth embodiment, the present invention provides a method according to the fortieth embodiment, wherein the at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In a fifty-sixth embodiment, the present invention provides a method according to the fortieth embodiment, wherein the at least one bioactive agent includes at least one type of functional cell.

In a fifty-seventh embodiment, the present invention provides a method according to the fifty-sixth embodiment, wherein applying includes administering approximately 1 million cells or less to the at least one channel at the target site.

In a fifty-eighth embodiment, the present invention provides a method of directing at least one bioactive agent to a particular site within a subject, comprising: identifying a particular site within a subject at which to direct at least one bioactive agent present in the subject, applying laser light to the identified particular site, and creating at least one channel with laser light at the identified site.

In a fifty-ninth embodiment, the present invention provides a method according to the fifty-eighth embodiment, wherein creating at least one channel with laser light causes a reaction in the subject that promotes migration of the one or more bioactive agent to the identified site.

In a sixtieth embodiment, the present invention provides a method according to the fifty-eighth embodiment, wherein applying laser light and creating at least one channel are sufficient to create an injury response at least 250 microns below the surface of the tissue at the identified site.

In a sixth-first embodiment, the present invention provides a method according to the sixtieth embodiment, wherein the injury response is a laser-generated injury response.

In a sixty-second embodiment, the present invention provides a method according to the fifty-eighth embodiment, wherein creating at least one channel includes creating at least one channel having a predetermined depth of at least 300 microns as measured from a surface of the identified site.

In a sixty-third embodiment, the present invention provides a method according to the sixty-second embodiment, wherein creating at least one channel includes creating at least one channel having a predetermined depth in the range of about 300 to 2000 microns as measured from the surface of the identified site.

In a sixty-fourth embodiment, the present invention provides a method according to the fifty-eighth embodiment, wherein the at least one bioactive agent is circulating in the subject.

In a sixty-fifth embodiment, the present invention provides a method according to the fifty-eighth embodiment, further comprising introducing the at least one bioactive agent into the subject.

In a sixty-sixth embodiment, the present invention provides a method according to the sixty-fifth embodiment, wherein introducing the at least one bioactive agent occurs prior to creating at least one channel with laser light at the identified site.

In a sixty-seventh embodiment, the present invention provides a method according to the sixty-fifth embodiment, wherein introducing the at least one bioactive agent occurs by injection.

In a sixty-eighth embodiment, the present invention provides a method according to the fifty-eighth embodiment, wherein the at least one bioactive agent includes at least one type of functional cell.

In a sixty-ninth embodiment, the present invention provides a method according to the fifty-eighth embodiment, wherein the at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In a seventieth embodiment, the method according to the fifty-eighth embodiment further comprises creating a plurality of channels at the identified particular site with the laser light.

In a seventy-first embodiment, the present invention provides a method according to the fifty-eighth embodiment, wherein applying laser light includes applying ablative laser light.

In a seventy-second embodiment, the present invention provides a method according to the seventy-first embodiment, wherein applying laser light includes applying fractional ablative laser light.

In a seventy-third embodiment, the present invention provides a laser-assisted method of treating a systemic condition, comprising: applying laser light to a target site of a subject, wherein the target site is spaced apart from a site affected by the condition to be treated, creating at least one channel of a predetermined depth at the target site using the laser, administering at least one bioactive agent to the target site of the subject at said channel for migration into said channel and systemic delivery to the affected site.

In a seventy-forth embodiment, the present invention provides a method according to the seventy-third embodiment, wherein creating at least one channel with laser light causes a reaction in the subject that promotes systemic migration of the one or more bioactive agent.

In a seventy-fifth embodiment, the present invention provides a method according to the seventy-third embodiment, wherein applying laser light and creating at least one channel are sufficient to create an injury response at least 250 microns below the surface of the tissue of the target site.

In a seventy-sixth embodiment, the present invention provides a method according to the seventy-fifth embodiment, wherein the injury response is a laser-generated injury response.

In a seventy-seventh embodiment, the present invention provides a method according to the seventy-third embodiment, wherein the predetermined depth of the at least one channel is at least 300 microns as measured from a surface of the tissue at the target site.

Bone marrow was withdrawn from a donor pig and mesenchymal stem cells (MSC) were established by routine methods known by those skilled in the art. Donor MSC's were then transduced with lentivirus containing expression vector(s) comprising a yellow fluorescent protein (YFP) nucleic acid sequence resulting in protein expression of YFP exclusively in the nucleus of transduced MSC cells. The transduction vector(s) also contained a non-expressed sequence unique to the vector for later use in molecular tracking of the transduced MSC.

In a seventy-eighth embodiment, the present invention provides a method according to the seventy-seventh embodiment, wherein the predetermined depth of the at least one channel is in the range of about 300 to 2000 microns as measured from the surface of the tissue at the target site.

In a seventy-ninth embodiment, the present invention provides a method according to the seventy-third embodiment, wherein the tissue at the target site is skin.

In an eightieth embodiment, the present invention provides a method according to the seventy-ninth embodiment, wherein creating at least one channel includes creating at least one channel having a depth at least sufficient to penetrate the dermal layer of skin.

In an eighty-first embodiment, the present invention provides a method according to the seventy-third embodiment, wherein applying a laser includes applying ablative laser light to the target site.

In an eighty-second embodiment, the present invention provides a method according to the eighty-first embodiment, wherein applying a laser includes applying fractional ablative laser.

In an eighty-third embodiment, the present invention provides a method according to the seventy-third embodiment, wherein the condition to be treated is at least one of tissue damage, organ damage, myocardial infarction, chronic tissue disease, chronic lung disease, reduced immune function, reduced hematopoietic function, vascular disorders, artery disease, stroke, lymphedema, carcinoma, tumor, organ loss, partial organ loss, and tissue loss.

In an eighty-fourth embodiment, the present invention provides a method according to the seventy-third embodiment, wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In an eighty-fifth embodiment, the present invention provides a method according to the seventy-third embodiment, wherein the at least one bioactive agent comprises at least one type of functional cells.

In an eighty-sixth embodiment, the present invention provides a method according to the seventy-third embodiment, wherein the at least one bioactive agent includes at least a vaccine for vaccination against a particular and predetermined antigen.

In an eighty-seventh embodiment, the present invention provides the use of at least one bioactive agent applied to an opening of at least one channel created by laser ablation for treating a systemic condition.

In an eighty-eighth embodiment, the present invention provides a use as recited in the eighty-seventh embodiment, wherein the systemic condition is at least one of tissue damage, organ damage, myocardial infarction, chronic tissue disease, chronic lung disease, reduced immune function, reduced hematopoietic function, vascular disorders, artery disease, stroke, lymphedema, carcinoma, tumor, organ loss, partial organ loss, and tissue loss.

In an eighty-ninth embodiment, the present invention provides a use as recited in the eighty-seventh embodiment, wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In a ninetieth embodiment, the present invention provides a use as recited in the eighty-seventh embodiment, wherein said at least one bioactive agent comprises at least one type of functional cells.

In a ninety-first embodiment, the present invention provides a use as recited in the ninetieth embodiment, wherein said at least one bioactive agent comprises a mixture of cells.

In a ninety-second embodiment, the present invention provides a use as recited in the ninety-first embodiment, wherein said mixture of cells comprises a heterogenous mixture of cells.

In a ninety-third embodiment, the present invention provides a use as recited in the eighty-seventh embodiment, wherein said at least one bioactive agent comprises approximately 1 million cells or less administered to the at least one channel.

In a ninety-fourth embodiment, the present invention provides the use of at least one bioactive agent applied to an opening of at least one channel created by laser ablation for vaccination against a particular and predetermined antigen.

In a ninety-fifth embodiment, the present invention provides a use as recited in the ninety-fourth embodiment, wherein said at least one bioactive agent comprises a vaccine.

In a ninety-sixth embodiment, the present invention provides a method of stimulating endogenous stem cell production comprising: identifying a target site on a subject, applying laser light to the identified target site, creating at least one channel at the identified target site with the laser light, and administering at least one bioactive agent to the at least one channel, wherein the at least one bioactive agent includes at least one type of functional cell.

In a ninety-seventh embodiment, the present invention provides a method according to the ninety-sixth embodiment, wherein the at least one bioactive agent includes at least one type of stem cell.

In a ninety-eighth embodiment, the present invention provides a method according to the ninety-seventh embodiment, wherein the at least one type of bioactive agent includes mesenchymal stem cells.

In a ninety-nineth embodiment, the present invention provides a method according to the ninety-sixth embodiment, wherein administering includes administering approximately 1 million functional cells or less to the at least one channel at the target site.

In a one hundredth embodiment, the present invention provides a method according to the ninety-sixth embodiment, wherein the at least one type of functional cell applied to the channel is the same type of cell as the endogenous cells whose circulation is stimulated.

In a one hundred and first embodiment, the present invention provides a method according to the ninety-sixth embodiment, wherein applying laser light and creating at least one channel are sufficient to create an injury response at least 250 microns below the surface of the tissue of the target site.

In a one hundred and second embodiment, the present invention provides a method according to the one hundred and first embodiment, wherein the injury response is a laser-generated injury response.

In a one hundred and third embodiment, the present invention provides a method according to the ninety-sixth embodiment, wherein creating at least one channel includes creating at least one channel having a predetermined depth of at least 300 microns as measured from a surface of the tissue at the target site.

In a one hundred and fourth embodiment, the present invention provides a method according to the one hundred and third embodiment, wherein creating at least one channel includes creating at least one channel having a predetermined depth in the range of about 300 to 2000 microns as measured from the surface of the tissue at the target site.

In a one hundred and fifth embodiment, the present invention provides a method according to the ninety-sixth embodiment, wherein the tissue at the target site is skin.

In a one hundred and sixth embodiment, the present invention provides a method according to the one hundred and fifth embodiment, wherein creating at least one channel includes creating at least one channel having a depth at least sufficient to penetrate the dermal layer of skin.

In a one hundred and seventh embodiment, the present invention provides a method according to the ninety-sixth embodiment, wherein applying laser light includes applying ablative laser light to the identified target site.

In a one hundred and eighth embodiment, the present invention provides a method according to the one hundred and seventh embodiment, wherein applying laser light includes applying fractional ablative laser light to the identified target site.

In a one hundred and ninth embodiment, the present invention provides a method according to the ninety-sixth embodiment, further comprising creating a plurality of channels at the identified target site with the laser light.

In a one hundred and tenth embodiment, the present invention provides a method according to the one hundred and ninth embodiment, wherein administering at least one bioactive agent comprises administering the at least one bioactive agent to the plurality of channels at the identified target site.

In a one hundred and eleventh embodiment, the present invention provides a method according to the ninety-sixth embodiment, wherein creating at least one channel at the identified target site with the laser light further comprises creating said at least one channel at the identified target site with the laser light to cause a laser-generated injury within said at least one channel at which said at least one bioactive agent is administered, said laser-generated injury in said at least one channel at which said at least one bioactive agent is administered stimulating the release of a greater number of endogenous stem cells into circulation in the subject than circulate under non-laser generated injury conditions.

In a one hundred and twelfth embodiment, the present invention provides a method according to the ninety-sixth embodiment, wherein creating at least one channel at the identified target site with the laser light further comprises creating said at least one channel at the identified target site with the laser light to cause a laser-generated injury within said at least one channel at which said at least one bioactive agent is administered, said laser-generated injury in said at least one channel at which said at least one bioactive agent is administered stimulating the growth of endogenous stem cells circulating in the subject than grow in circulation under non-laser generated injury conditions.

In a one hundred and thirteenth embodiment, the present invention provides a method according to the ninety-sixth embodiment, further comprising harvesting endogenous stem cells from blood circulating in the subject.

In a one hundred and fourteenth embodiment, the present invention provides a system for delivery of a bioactive agent, comprising: a laser capable of producing laser light for creating at least one channel of a predetermined depth in a target tissue at a target site, thereby defining an opening at the surface of the target tissue, and at least one bioactive agent disposable at the opening of the at least one channel and capable of migration into and through the at least one channel and systemic delivery to a distal site for beneficial effect at the distal site.

In a one hundred and fifteenth embodiment, the present invention provides a system according to the one hundred and fourteenth embodiment, wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In a one hundred and sixteenth embodiment, the present invention provides a system according to the one hundred and fourteenth embodiment, wherein said at least one bioactive agent includes at least one type of functional cell.

In a one hundred and seventeenth embodiment, the present invention provides a system according to the one hundred and fourteenth embodiment, wherein said at least one bioactive agent includes about 1 million functional cells or less.

In a one hundred and eighteenth embodiment, the present invention provides a system according to the one hundred and fourteenth embodiment, wherein said at least one bioactive agent includes at least one type of stem cell.

In a one hundred and nineteenth embodiment, the present invention provides a system according to the one hundred and eighteenth embodiment, wherein said at least one bioactive agent includes mesenchymal stem cells.

In a one hundred and twentieth embodiment, the present invention provides a system according to the one hundred and fourteenth embodiment, wherein said at least one bioactive agent comprises a mixture of cells.

In a one hundred and twenty-first embodiment, the present invention provides a system according to the one hundred and twentieth embodiment, wherein said mixture of cells comprises a heterogenous mixture of cells.

In a one hundred and twenty-second embodiment, the present invention provides a system according to the one hundred and fourteenth embodiment, wherein said at least one channel is a coagulated channel.

In a one hundred and twenty-third embodiment, the present invention provides a system according to the one hundred and fourteenth embodiment, further comprising a plurality of channels of predetermined depths in said target tissue, wherein said plurality of channels collectively define a matrix.

In a one hundred and twenty-fourth embodiment, the present invention provides a system according to the one hundred and fourteenth embodiment, wherein said laser is an ablative laser.

In a one hundred and twenty-fifth embodiment, the present invention provides a system according to the one hundred and twenty-fourth embodiment, wherein said laser is a fractional ablative laser.

In a one hundred and twenty-sixth embodiment, the present invention provides a system according to the one hundred and fourteenth embodiment, wherein said laser is capable of creating channels having a predetermined depth of at least 300 microns.

In a one hundred and twenty-seventh embodiment, the present invention provides a system according to the one hundred and twenty-sixth embodiment, wherein said laser is capable of creating channels having a predetermined depth in the range of about 300 to 2000 microns.

In a one hundred and twenty-eighth embodiment, the present invention provides a system according to the one hundred and fourteenth embodiment, wherein said laser is capable of creating an injury response at least 250 microns below the surface of the tissue of the target site.

In a one hundred and twenty-ninth embodiment, the present invention provides a system according to the one hundred and twenty-eighth embodiment, wherein the injury response is a laser-generated injury response.

In a one hundred and thirtieth embodiment, the present invention provides a system according to the one hundred and fourteenth embodiment, wherein said laser is capable of creating at least one channel having a width of about 400 microns or less.

In a one hundred and thirty-first embodiment, the present invention provides a system according to the one hundred and thirtieth embodiment, wherein said laser is capable of creating at least one channel having a width of about 200 microns or less.

In a one hundred and thirty-second embodiment, the present invention provides a system according to the one hundred and thirty-first embodiment, wherein said laser is capable of creating at least one channel having a width in the range of about 10 to 200 microns.

In a one hundred and thirty-third embodiment, the present invention provides a system according to the one hundred and fourteenth embodiment, wherein said laser is capable of creating a plurality of channels of predetermined depths in the tissue at the target site.

In a one hundred and thirty-fourth embodiment, the present invention provides a system according to the one hundred and thirty-third embodiment, wherein said laser is capable of creating a plurality of channels in the range of about 400 to 800 channels per centimeter square of target site.

In a one hundred and thirty-fifth embodiment, the present invention provides a kit for the delivery of at least one bioactive agent, comprising: at least one bioactive agent, and instructions for application of said at least one bioactive agent to at least one laser-created channel in a target tissue for systemic delivery of said bioactive agent to a distal site spaced apart from the target tissue.

In a one hundred and thirty-sixth embodiment, the present invention provides a kit according to the one hundred and thirty-fifth embodiment, wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In a one hundred and thirty-seventh embodiment, the present invention provides a kit according to the one hundred and thirty-fifth embodiment, wherein said at least one bioactive agent comprises at least one type of functioning cells.

In a one hundred and thirty-eighth embodiment, the present invention provides a kit according to the one hundred and thirty-seventh embodiment, wherein said at least one bioactive agent includes at least one type of stem cell.

In a one hundred and thirty-ninth embodiment, the present invention provides a kit according to the one hundred and thirty-seventh embodiment, wherein said at least one bioactive agent comprises a mixture of a first type of functioning cell and at least a second type of functioning cell.

In a one hundred and fortieth embodiment, the present invention provides a kit according to the one hundred and thirty-fifth embodiment, further comprising a laser capable of producing laser light to create said at least one channel.

In a one hundred and forty-first embodiment, the present invention provides a kit according to the one hundred and fortieth embodiment, wherein said laser comprises an ablative laser.

In a one hundred and forty-second embodiment, the present invention provides a kit according to the one hundred and fortieth embodiment, wherein said laser comprises a fractional ablative laser.

In a one hundred and forty-third embodiment, the present invention provides a kit according to the one hundred and fortieth embodiment, wherein said laser is mobile.

In a one hundred and forty-fourth embodiment, the present invention provides a kit according to the one hundred and fortieth embodiment, wherein said laser is hand-held.

In a one hundred and forty-fifth embodiment, the present invention provides a kit according to the one hundred and thirty-fifth embodiment, further comprising a tissue explant.

In a one hundred and forty-sixth embodiment, the present invention provides a kit according to the one hundred and forty-fifth embodiment, further comprising instructions for application of said at least one bioactive agent to said tissue explant to form a seeded explant and implantation of said seeded tissue explant within a subject.

In a one hundred and forty-seventh embodiment, the present invention provides a method of delivering a bioactive agent to a subject, comprising: applying laser light to a tissue explant, creating at least one channel of a predetermined depth in the tissue explant with the laser light, administering at least one bioactive agent to the tissue explant to obtain a seeded tissue explant, and implanting the seeded tissue explant into a subject.

In a one hundred and forty-eighth embodiment, the present invention provides a method according to the one hundred and forty-seventh embodiment, wherein the at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In a one hundred and forty-ninth embodiment, the present invention provides a method according to the one hundred and forty-seventh embodiment, wherein the at least one bioactive agent comprises at least one type of functional cells.

In a one hundred and fiftieth embodiment, the present invention provides a method according to the one hundred and forty-ninth embodiment, wherein the at least one bioactive agent comprises at least one type of stem cell.

In a one hundred and fifty-first embodiment, the present invention provides a method according to the one hundred and forty-seventh embodiment, wherein implanting comprises implanting the seeded tissue explant into a tissue of the subject.

In a one hundred and fifty-second embodiment, the present invention provides a method according to the one hundred and forty-seventh embodiment, wherein implanting comprises implanting the seeded tissue explant into an organ of the subject.

In a one hundred and fifty-third embodiment, the present invention provides a method according to the one hundred and forty-seventh embodiment, wherein implanting comprises implanting the seeded explant at an implantation site to a predetermined depth within the subject so as to enable systemic movement of the at least one bioactive agent from the seeded explant to sites within the subject distant from the implantation site.

In a one hundred and fifty-fourth embodiment, the present invention provides a method according to the one hundred and forty-seventh embodiment, wherein implanting occurs at a surface of the subject.

In a one hundred and fifty-fifth embodiment, the present invention provides a method according to the one hundred and forty-seventh embodiment, wherein implanting occurs subcutaneously.

In a one hundred and fifty-sixth embodiment, the present invention provides a method according to the one hundred and forty-seventh embodiment, wherein applying laser light includes applying ablative laser light.

In a one hundred and fifty-seventh embodiment, the present invention provides a method according to the one hundred and fifty-sixth embodiment, wherein applying laser light includes applying fractional ablative laser light.

In a one hundred and fifty-eighth embodiment, the present invention provides a method according to the one hundred and forty-seventh embodiment, further comprising creating a plurality of channels in the tissue explant.

In a one hundred and fifty-ninth embodiment, the present invention provides a method according to the one hundred and forty-seventh embodiment, wherein the at least one bioactive agent includes at least one corrective gene or corrective gene product capable of providing therapeutic benefit for a condition resulting from a corresponding aberrant gene or aberrant gene product.

In a one hundred and sixtieth embodiment, the present invention provides a method according to the one hundred and fifty-ninth embodiment, wherein the corrective gene product comprises RNA or protein.

In a one hundred and sixty-first embodiment, the present invention provides a method according to the one hundred and fifty-ninth embodiment, wherein the at least one bioactive agent comprises cells having at least one corrective gene or corrective gene product and capable of expressing the corrective gene or corrective gene product.

In a one hundred and sixty-second embodiment, the present invention provides a system for delivering bioactive agent to tissue, comprising: a tissue explant, at least one channel disposed throughout said tissue explants and having an opening defined at a surface of said tissue explant, and at least one bioactive agent applied to said opening of said at least one channel.

In a one hundred and sixty-third embodiment, the present invention provides a method according to the one hundred and sixty-second embodiment, wherein said tissue explant is of biologic origin.

In a one hundred and sixty-fourth embodiment, the present invention provides a method according to the one hundred and sixty-second embodiment, wherein said tissue explant is synthetic.

In a one hundred and sixty-fifth embodiment, the present invention provides a method according to the one hundred and sixty-second embodiment, wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

In a one hundred and sixty-sixth embodiment, the present invention provides a method according to the one hundred and sixty-second embodiment, wherein said at least one bioactive agent comprises at least one type of functional cells.

In a one hundred and sixty-seventh embodiment, the present invention provides a method according to the one hundred and sixty-sixth embodiment, wherein said at least one bioactive agent comprises at least one type of stem cell.

In a one hundred and sixty-eighth embodiment, the present invention provides a method according to the one hundred and sixty-second embodiment, further comprising a plurality of channels disposed throughout said tissue explant so as to define a matrix of channels.

In a one hundred and sixty-ninth embodiment, the present invention provides a method according to the one hundred and sixty-second embodiment, further comprising a laser capable of producing laser light to create said at least one channel in said tissue explant.

In a one hundred and seventieth embodiment, the present invention provides a method according to the one hundred and sixty-second embodiment, wherein said laser is an ablative laser.

In a one hundred and seventy-first embodiment, the present invention provides a method according to the one hundred and seventieth embodiment, wherein said laser is a fractional ablative laser.

In a one hundred and seventy-second embodiment, the present invention provides the use of a tissue explant including at least one laser ablated channel and at least one bioactive agent applied to said at least one laser ablated channel for treating a systemic condition.

In a one hundred and seventy-third embodiment, the present invention provides a method of treating a biofilm on a subject comprising applying laser light to an affected site on the subject sufficient to disrupt the biofilm.

In a one hundred and seventy-fourth embodiment, the present invention provides a method according to the one hundred and seventy-third embodiment, further comprising administering at least one bioactive agent to the affected site.

In a one hundred and seventy-fifth embodiment, the present invention provides a method according to the one hundred and seventy-fourth embodiment, wherein the at least one bioactive agent comprises one of cells, protein, peptide, peptide fragment, nucleic acid, nucleotide fragment, gene, pharmaceutical compound, therapeutic compound, medicament, small molecule, aptamer, and combinations thereof.

In a one hundred and seventy-sixth embodiment, the present invention provides a method according to the one hundred and seventy-fifth embodiment, wherein the at least one bioactive agent is an antimicrobial agent.

In a one hundred and seventy-seventh embodiment, the present invention provides a method according to the one hundred and seventy-fifth embodiment, wherein the at least one bioactive agent is at least one type of functional cell capable of expressing an antimicrobial agent.

In a one hundred and seventy-eighth embodiment, the present invention provides a method according to the one hundred and seventy-fourth embodiment, further comprising applying a subsequent round of laser treatment to the affected site.

In a one hundred and seventy-ninth embodiment, the present invention provides a method of treating a disease or condition in a subject in need thereof, comprising:
a. creating at least one channel in the tissue of the subject at a target site via the application of laser light; and
b. administering at least one bioactive agent to the at least one channel in an amount sufficient to treat the disease or condition.

In a one hundred and eightieth embodiment, the present invention provides a method according to the one hundred and seventy-ninth embodiment, wherein the at least one bioactive agent is selected from the group consisting of stem cells, proteins, peptides, peptide fragments, nucleic acid molecules, genes, pharmaceutical compounds, aptamers, and combinations thereof.

In a one hundred and eighty-first embodiment, the present invention provides a method according to the one hundred and eightieth embodiment, wherein the stem cells are selected from the group consisting of mesenchymal stem cells and lin(−) hematopoetic stem cells.

In a one hundred and eighty-second embodiment, the present invention provides a method according to the one hundred and seventy-ninth embodiment, wherein the disease or condition is located at the target site.

In a one hundred and eighty-third embodiment, the present invention provides a method according to the one hundred and seventy-ninth embodiment, wherein the disease or condition is at a location distal to the target site, and the at least one bioactive agent migrates to the distal location in order to treat the disease or condition.

In a one hundred and eighty-fourth embodiment, the present invention provides a method according to the one hundred and seventy-ninth embodiment, wherein the at least one channel has a diameter of about 120 to about 430 microns and a depth from about 300 to about 3000 microns.

In a one hundred and eighty-fifth embodiment, the present invention provides a method according to the one hundred and seventy-ninth embodiment, wherein the at least one channels are created at a density of about 400 to about 800 channels per square centimeter.

In a one hundred and eighty-sixth embodiment, the present invention provides a method according to the one hundred and seventy-ninth embodiment, wherein the target site is the skin of the subject.

In a one hundred and eighty-seventh embodiment, the present invention provides a method according to the one hundred and seventy-ninth embodiment, wherein the application of laser light also induces an injury response in the target site.

In a one hundred and eighty-eighth embodiment, the present invention provides a method according to the one hundred and seventy-ninth embodiment, wherein the application of laser light causes cells to migrate to the target site.

In a one hundredth and eighty-ninth embodiment, the present invention provides a method of treating a disease or condition in a subject in need thereof using a tissue explant, comprising:
a. creating at least one channel in the tissue explant via the application of laser light;
b. administering the at least one bioactive agent to the at least one channel; and
c. transplanting the tissue explant at a target site in the subject.

In a one hundred and ninetieth embodiment, the present invention provides a method according to the one hundred and eighty-ninth embodiment, wherein the at least one bioactive agent is selected from the group consisting of stem cells, proteins, peptides, peptide fragments, nucleic acid molecules, genes, pharmaceutical compounds, aptamers, and combinations thereof.

In a one hundred and ninety-first embodiment, the present invention provides a method according to the one hundred and ninetieth embodiment, wherein the stem cells are selected from the group consisting of mesenchymal stem cells and lin(−) hematopoetic stem cells.

In a one hundred and ninety-second embodiment, the present invention provides a method according to the one hundred and eighty-ninth embodiment, wherein the disease or condition is located at the target site.

In a one hundred and ninety-third embodiment, the present invention provides a method according to the one hundred and eighty-ninth embodiment, wherein the disease or condition is at a location distal to the target site, and the at least one bioactive agent migrates to the distal location in order to treat the disease.

In a one hundred and ninety-fourth embodiment, the present invention provides a method according to the one hundred and eighty-ninth embodiment, wherein the at least one channel has a diameter of about 120 to about 430 microns and a depth from about 300 to about 3000 microns.

In a one hundred and ninety-fifth embodiment, the present invention provides a method according to the one hundred and eighty-ninth embodiment, wherein the at least one channels are created at a density of about 400 to about 800 channels per square centimeter.

In a one hundred and ninety-sixth embodiment, the present invention provides a method according to the one hundred and eighty-ninth embodiment, wherein the target site is the skin of the subject.

In a one hundred and ninety-seventh embodiment, the present invention provides a method according to the one hundred and eighty-ninth embodiment, wherein the application of laser light also induces an injury response in the target site.

In a one hundred and ninety-eighth embodiment, the present invention provides a method according to the one hundred and eighty-ninth embodiment, wherein the application of laser light causes cells to migrate to the target site.

### BRIEF DESCRIPTION OF THE DRAWINGS

For a fuller understanding of the nature of the present invention, reference should be had to the following detailed description taken in connection with the accompanying figures in which:

FIG. 1 shows photographs of burn wounds of porcine dorsal skin 35 days after treatment with laser ablation and mesenchymal stem cells (MSC) transduced with yellow fluorescent protein (YFP) expressing lentivirus (panel A) and porcine dorsal skin covered with polyurethane film (no MSC delivery) to act as a control (panel B).

FIG. 2 shows a fluorescence microscopy image at 200× magnification of a full thickness wound at day 5 following laser ablation and application of MSC. Nuclei are stained with DAPI and the circles indicate transduced cells that dual fluoresce from both DAPI and YFP expressing nuclei. The path of laser ablation is outlined by the solid interconnecting lines.

FIG. 3 shows a fluorescence and DIC overlay image at 100× magnification of a burn wound on porcine dorsal skin at day 7 after being treated with laser ablation and MSC transduced with YFP expressing lentivirus. The labeled cells (indicated by arrows) are MSC cells expressing YFP.

FIG. 4 shows a fluorescent image at 400× magnification of acutely inflamed devitalized crust of a burn wound of porcine dorsal skin at day 7 after being treated with laser ablation and MSC transduced with YFP expressing lentivirus. The labeled cells (circled) represent MSC cells expressing YFP.

FIG. 5 shows full thickness burn wounds of porcine dorsal skin at 100× magnification 14 days after treatment with laser ablation and MSC transduced with YFP expressing lentivirus (panel A) and treatment with laser ablation and saline (control) (panel B). Stars in panel A illustrate fetal collagen like arrangement that is a histological marker of improved wound healing, having less scar like features.

FIG. 6 shows laser+MSC treated burn wounds (panel A) and laser+saline treated burn wounds (panel B) 7 days after treatment at 100× magnification. Black lines highlight the decreased depth of the ablated channels at day 7 post treatment in the MSC treated group indicative of faster healing.

FIG. 7 shows a gel of PCR amplification of lentivirus specific gene. Lane 1 contains 100 base pair (bp) marker; lane 2 contains non-template control; lane 3 contains control porcine genomic DNA from bone marrow (source 1); lane 4 contains control porcine genomic DNA from bone marrow (source 2); lane 5 contains laser treated pig genomic DNA from bone marrow; lane 6 contains control human genomic DNA; and lane 7 contains the positive control virus plasmid DNA. Lane 5 shows a band at approximately 384 bp, illustrating the presence of lentivirus, and therefore transduced MSC's, in bone marrow.

FIG. 8 shows a gel of PCR amplification of lentivirus specific gene. Lane 1 contains the lentiviral gene control; lane 2 contains laser treated pig genomic DNA from bone marrow; and lane 3 contains a 1 kilobase (Kb) marker. Lane 2 illustrates presence of transduced MSC gene (arrow) in bone marrow.

FIG. 9 shows fluorescence-activated cell sorting (FACS) analysis of blood adjusted to look for the presence of circulating cells expressing green fluorescent protein (GFP) at week 3 following treatment. The presence of any circulating GFP positive cells indicates that hematopoietic stem cells had been delivered with fractional laser through the skin, entered the systemic circulation, engrafted into the bone marrow and remained functionally intact to reconstitute the hematopoietic system.

FIG. 10 shows a fluorescent image of a wound at day 14 after being treated with fractional laser and sterile saline but not MSCs, and which is distant from a first site which was treated with fractional laser and MSC transduced with YFP expressing lentivirus. The arrow illustrates a blood vessel. The circle highlights a labeled MSC originally delivered by fractional laser at a target site, but which is now located at the second distant lasered site. This indicates both the ability of fractional laser to deliver stem cells into the systemic circulation and that fractional laser (here without administering cells directly) can be used to attract circulating stem cells to a distant site.

FIG. 11 shows the crust over a full thickness wound treated with laser+MSC at 400× magnification on day 14 post treatment. The circle shows a labeled MSC that is dividing. This indicates that cells delivered via fractional laser remain functional.

FIG. 12 shows photographs of C57/BL6 mice treated with ionizing radiation to inhibit hair growth and treated with either fractional laser treatment and lineage negative syngeneic bone marrow cells (panel A), fractional laser treatment and total syngeneic bone marrow cells (panel B), or fractional laser treatment alone (panel C), all at four weeks after treatment. Arrows in panels A and B indicate that treatment with laser and cells produces dramatic hair regrowth compared to the negative control in panel C in which no cells were delivered.

FIG. 13 is a graphical representation depicting the reduction of methicillin resistant Staphylococcus aureus (MRSA) biofilms upon application of laser treatment plus gentamycin, as compared to gentamycin or laser treatment alone.

FIG. 14 is a graphical representation of the number of endogenous stem cells circulating in the blood of porcine subjects with second degree burns and treated with laser ablation and labeled mesenchymal stem cells (MSCs) at various time points over the course of three weeks. The top line (square data points) shows endogenous MSC levels in the blood when allogenic labeled MSCs were delivered with laser ablation. The middle line (circular data points) shows endogenous MSC levels in the blood when autologous labeled MSCs were delivered with laser ablation. The bottom line (triangular data points, along the x-axis) shows endogenous MSC levels in the blood with only laser ablation.

FIG. 15 shows porcine burn wounds treated with fractional laser alone (A) or with fractional laser plus MSCs (B). There is significantly less inflammation in the burn treated with MSCs. The inflammatory infiltrate in the MSC treated wound in more monocytic with many less polymorphonuclear leukocytes (PMNs). MSCs delivered by laser have significantly reduced the inflammatory component within the burn wound.

FIG. 16 shows photographs of C57/BL6 mice treated with ionizing radiation to inhibit hair growth and given MSCs by intravenous (IV) injection, and treated with fractional laser treatment. Hair regrowth is notable where laser treatment was applied, indicating an ability to direct circulating cells to a particular site to produce a beneficial effect.

### DETAILED DESCRIPTION

The present invention is directed to methods of laser assisted delivery of bioactive agent(s) locally and systemically in subjects, as well as uses, systems, and kits for same. Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting of the invention. As used herein, the indefinite articles “a”, “an” and “the” should be understood to include plural reference unless the context clearly indicates otherwise.

The phrase “and/or,” as used herein, should be understood to mean “either or both” of the elements so conjoined, i.e., elements that are conjunctively present in some cases and disjunctively present in other cases. As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

As used herein, the terms “including”, “includes”, “having”, “has”, “with”, or variants thereof, are intended to be inclusive similar to the term “comprising.”

As used herein, the term “subject” refers to any animal (e.g., mammals, birds, reptiles, amphibians, fish), including, but not limited to, humans, non-human primates, rodents, swine, canine, feline and the like, which is to be the recipient of the present invention. Typically, the terms “subject” and “patient” may be used interchangeably herein in reference to a subject; however, it is contemplated that a subject would not necessarily be a patient under hospital and/or physician care.

As used herein, the term “bioactive agent” refers to any cell or biological compound or chemical compound that has an effect on living cells or tissues. A bioactive agent comprises, but is not limited to, cells, nucleic acid sequence such as of DNA or RNA, protein, peptides, nucleotide fragments, genes, pharmaceutical compositions, medicaments, chemical compounds, small molecules, aptamers (including DNA, RNA, or peptide aptamers), and therapeutics. The bioactive agent may be in an isolated form, as isolated and purified from an animal, plant, bacterial, viral or other source. The bioactive may be synthetically created or derived. In some embodiments, the bioactive agent may be a polypeptide, polynucleotide, or fragment thereof, and may be recombinant or isolated.

In some embodiments, the bioactive agent may be at least one cell, and preferably at least functional cell, or a population of such cells. Any type of functional cell is contemplated herein as a bioactive agent; however some examples include stem cells, mesenchymal stem cells, bone marrow stem cells, progenitor cells, bone marrow progenitor cells, lymphocytes, immune cells, immune modulation cells, mature or adult cells, etc. The cell may be of ectodermal, mesodermal or endodermal origin. In particular embodiments in which the cell is a stem cell, the stem cell may have any range of potency or differentiation potential. For example, the stem cells may be may be pluripotent, multipotent, or totipotent. Further, the stem cells may be dedicated stem cells which are unipotent, such as a muscle stem cell, or can be partially or fully induced or differentiated stem cells. The stem cells may also be adult cells that have been de-differentiated to a more multipotent form, or may be embryonic or near-embryonic stem cells, such as derived from umbilical cord or like matter. In other embodiments, the cell(s) may be progenitor cells, which can have varying ranges of potencies, including pluripotent, multipotent, and totipotent, but are limited in the number of cellular divisions possible. This is in contrast to stem cells which also can have different potencies but are perpetually self-renewing. In still other embodiments, the cells may be mature cells that express mature cell markers. The at least one cell may be autologous in which it is derived or taken from the same individual as is the subject of the present invention. In other embodiments, the cell(s) may be allogenic, being derived from a different individual of the same species as the subject of the present invention. In still other embodiments, the cell(s) may be of a different species than the subject. Moreover, the at least one cell may be applied to the subject in its native form, or it may be genetically and/or molecularly engineered prior to application to the subject. For instance, the bioactive agent may be transduced, transformed, transfected, infected, or otherwise genetically or molecularly engineered. The bioactive agent may be a cell or cell line engineered to include a particular vector, such as an expression vector, mammalian expression vector, viral vector, etc. which may include a transgene and/or be capable of producing a particular RNA or protein. The bioactive agent (s) may be a single type of any of the above, or a combination or mixture of any of the above.

As used herein, the term “fragment” refers to a portion of a compound. For example, when referring to a protein, a fragment is a plurality of consecutive amino acids comprising less than the entire length of the polypeptide. When referring to DNA, RNA, or a gene, a fragment is a plurality of consecutive nucleic acids comprising less than the entire length thereof, such as an oligonucleotide.

As used herein, the term “administering” refers to providing an effective amount of a bioactive agent to a subject to render the desired biological response, benefit, or therapeutic outcome. The bioactive agent of the present invention can be administered alone or with other compounds, excipients, fillers, binders, carriers, solvents, or other vehicles selected based upon the chosen route of administration and standard pharmaceutical practice. Administration may be by way of carriers or vehicles, such as solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; drops; other proteins and peptides; synthetic polymers; microspheres; nanoparticles; and the like.

Administering includes contacting a local or target site or applying a bioactive agent onto the local or target site on the subject being treated. The term “contacting” refers to actions directed to the creation of a spatial relationship between the cell(s) at the opening of a laser-generated channel and the bioactive agent(s) (or vehicle containing the bioactive agent(s)), provided for a predetermined and specified time and under conditions appropriate to render a desired biological response in the contacted cell(s)/tissue(s) or systemically within the subject being treated. “Systemic” responses may include the bioactive agent entering the cell or tissue at the site of administration and generating a response and/or localizing itself in another part of the subject's body. For instance, upon entry at the site of administration, the bioactive agent may be circulated (such as through circulatory or lymphatic systems), dispersed, recruited, directed, or otherwise migrate within the subject's body for action and affect at sites distal to, or separate from, the site of administration. The spatial relationship between the cell(s) or tissue(s) and the bioactive agent(s) can include direct contact, whereby the agent elicits a response on the contacted cell or tissue surface directly or enters the cell or tissue for further action, or indirect contact, whereby the agent elicits a response on the cell through extracellular signaling (e.g., following activation or modification of another substance which interacts with the contacted cell or tissue). As applied herein, a “biological response” includes any change or alteration in the biology, chemistry, biochemistry, or physiology of the cell(s) or tissue(s), such as but not limited to an arrest, inhibition, reduction, slowing, or regression of a disorder or condition, and/or an increased, augmented, enhanced, stimulated, or restored function or process. Examples include treatment of a disease, disorder, or condition, which may be acute or chronic, isolated or systemic; wound repair and healing, including burns; functional rescue or restoration; system modulation, such as immune modulation; bone marrow transplant; stimulation of stem cell production and release; hair growth and others as well.

The bioactive agent of the present invention may also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. Specifically, the pharmaceutically acceptable carriers, excipients, binders, and fillers contemplated for use in the practice of the present invention are those which render the compounds of the invention amenable to delivery as described herein. Moreover, the packaging material may be biologically inert or lack bioactivity, such as plastic polymers, silicone, etc., and may be processed internally by the subject without altering the effectiveness of the bioactive agent packaged and/or delivered therewith.

The term “effective amount,” as applied to the bioactive agents described herein means the quantity necessary to render the desired biological response or therapeutic result. For example, an effective amount is a level effective to treat, cure, alleviate, or reduce the symptoms of a disorder or condition for which the bioactive agent is being administered, or to cause an increase or decrease in particular biological, chemical, biochemical, or physiological activity as such as described above. Specific amounts needed to reach an effective amount depend upon a variety of factors including the particular biological response desired and scope or degree of change desired in relation to the current state of the subject; the disorder or condition being treated, if applicable, and its severity and/or stage of development/progression; the bioavailability and activity of the specific bioactive agent used; introduction site on the subject; the rate of systemic migration, if applicable; the rate of clearance of the bioactive agent and other pharmacokinetic properties; the duration of treatment; treatment regimen; drugs used in combination or coincident with the specific bioactive agent; the age, body weight, sex, diet, physiology and general health of the subject being treated; and like factors well known to one of skill in the relevant scientific art. Some variation will necessarily occur depending upon the condition of the subject being treated, and the physician or other individual administering treatment will, in any event, determine the appropriate dose for an individual subject. As used herein, “condition” refers to a disorder, disease or condition, or other departure from healthy or normal biological activity, and the terms can be used interchangeably. The terms would refer to any condition that impairs normal function. The condition may be caused by sporadic or heritable genetic abnormalities. The condition may also be caused by non-genetic abnormalities, such as from environmental influences. The condition may also be caused by injuries to a subject from environmental factors, such as, but not limited to, cutting, crushing, burning, piercing, stretching, shearing, injecting, or otherwise modifying a subject's cell(s), tissue(s), organ(s), system(s), or the like. Furthermore, the term “condition” encompasses wounds or lesions that form as a result of the injuries described herein, which may be a target for treatments also described herein. The disorder could also include biofilm formation on tissues and organs, such as in the case of biofilm formation over burn wounds.

As used herein, “treatment” or “treating” refers to arresting or inhibiting, or attempting to arrest or inhibit, the development or progression of a disorder or condition and/or causing, or attempting to cause, the reduction, suppression, regression, or remission of a disorder, condition and/or a symptom thereof. As would be understood by those skilled in the art, various clinical and scientific methodologies and assays may be used to assess the development or progression of a condition, and similarly, various clinical and scientific methodologies and assays may be used to assess the reduction, regression, or remission of a disorder, condition, or its symptoms.

In accordance with at least one embodiment of the present invention, a method of delivering at least one bioactive agent to a subject thereof comprises applying a laser, such as laser light, to a target site on the subject, creating at least one channel in the tissue of the target site with the laser, and administering one or more bioactive agents to the target site at the channel for migration into the channel to achieve a biological response. The at least one channel is deeper than traditionally created channels currently used for superficial treatments, and is sufficiently deep so as to cause a reaction in the body of the subject to promote migration of the bioactive agent throughout. The present method can therefore be used to deliver bioactive agent (s) locally, to the target site for action at the target site, or systemically to distal sites, as will be described in greater detail hereinafter.

As used herein, “target site” refers to an area to be treated by the methods of the invention that directly receives application of laser light and at which the channel(s) is created, as well as areas that are incident to, adjacent, and/or immediately surrounding the location of laser application and the resulting channel(s). The term “local site” may be used interchangeably with “target site.” The target or local site includes cells, extracellular matrix, fluids, and other matter of the tissue that is in direct contact with or immediately adjacent to the cells affected by the laser, as well as extending layers beyond the cells directly affected by the laser, for example by a distance of micrometers, millimeters, or centimeters. The target or local site encompasses not only surface cells and matter, such as the epidermis or outer layers of an organ in the case of open surgery, but also may include subcutaneous and internal cells and tissue as far as the bone marrow, depending on the length, depth, and placement of the channel created.

Moreover, the target site may be located on any tissue or organ of the body. For example, in at least one embodiment, the target site is located along the skin, such that the tissue affected by the laser at the target site is skin. However, in other embodiments, internal organs such as liver, muscle, heart, etc. may be the target site, and laser application may be facilitated by surgery (which may be open or laparoscopic), subcutaneous, transdermal, or other invasive or partially invasive techniques to permit direct application of laser light to the internal organ of choice, including the use of cannulas or other devices for directing the laser and/or laser light to the target site. As used herein, a “laser” refers to a high-energy beam of light that can be directed into certain areas or tissues. The beams of a laser are produced in one wavelength at a time and can vary in terms of the power or strength of the beam and the resulting tissue it can target. The sources of laser can be from ablative and non-ablative laser sources. A non-ablative laser has a lower energy level than an ablative laser and tends to cause damage to subsurface areas of the targeted tissue. An ablative laser has an intense energy that is used in bursts on the surface of the tissue being treated. The intense energy heats the water within the surface layers of the tissue, causing the water and tissue to vaporize. Each pass of the laser energy over the tissue causes the outermost layers to be removed in a precise and controlled way to an appropriate depth of penetration. A laser source can also be a fractional laser source. Fractional lasers only damage certain zones within a selected target area, producing a tiny dot or pixel-like area of treatment, hence only causing fractional damage from the heat of the light source. Fractional lasers can be ablative or non-ablative. The laser used in certain embodiments of the present invention includes an ablative fractional laser. For example, in some embodiments of the present invention, a fractional ablative Erbium-YAG (Er:YAG) laser is used, such as the Sciton Profractional XC having a spot size of 430 microns, a depth range 20-1500 microns, treatment densities 11% or 22%, and spot size 1-8. The Erbium-YAG laser is a tunable ablation laser with coagulation options, such as the ability to change coagulation on levels 0, 1, 2, 3, and specific tunable options. In other embodiments of the present invention, a Lumenis Ultrapulse laser, using a 10,600 nm wavelength and 240 watts, 225 mJ of energy, spot size of 120 microns, depth ranges from 75-1500 microns is used.

The beam of light created by the laser is adjustable for depth and diameter of the laser, and may be based on the software that controls the functioning and deployment of the laser. The channels created by the laser will have depth and diameter measurements corresponding to the laser light used to create the channel, discussed below. In some embodiments, the laser is used at depths of 500-1500 microns, such as with an Erbium-YAG laser.

In at least one other embodiment, the laser is used at depths ranging from 150-1500 microns, such as with a Deep FX (CO2) laser. The company has warnings and cautions for using this laser beyond 750 microns. By way of reference, one common current use of lasers is typical wrinkle treatments, which are only from 75-300 microns in depth. In many embodiments of the present invention, software is used to control the laser, such as the wavelength of light or energy used, the diameter and depth of channel created thereby, and whether and to what extent the channel will be coagulated.

In at least one embodiment, the software is designed to pass the laser in a scanning pattern over the target tissue, thus creating a plurality, or matrix, of channels therein. The space between the channels and/or the ratio of ablated tissue corresponding to channels and intervening non-lasered tissue defines a channel density. For example, the laser can be used to create approximately 400 channels per square centimeter. The laser may also be used to create a denser matrix of approximately 800 channels per square centimeter. It should be appreciated that these are merely examples, and are not meant to be strictly construed or limiting in any way, as the matrix or plurality of channels may include more or fewer channels than those given in the above examples.

As used herein, a “channel” refers to the area resulting from the controlled removal of cells, extracellular matrix, fluids, and other matter from a tissue by a laser. The channel can be any depth/length, and width, and is only limited by the capabilities of the laser and the desired treatment strategy or outcome. For example, the channel(s) can range from about 120-430 microns in diameter, and measure in the range up to 3000 microns in length or depth from the surface of the tissue, depending the type of laser used, the subject, and the area of treatment, among other factors.

For instance, in some embodiments, such as when using a CO2 ablative laser, the channels may have a diameter of about 120 microns. In other embodiments, such as using an Er:YAG laser, channels can have diameters of 430 microns. In some embodiments, each channel has a width or diameter of about 400 microns or less. Optimally, the channels have a width or diameter of about 200 microns or less, and in some embodiments may preferably be in the range of between 10 and 200 microns.

With regard to depths, in at least one embodiment, the channel(s) have depths of at least 300 microns as measured from the surface of the treated tissue, such as the target site. In some embodiments, the laser-created channel(s) have predetermined depths ranging between 300 and 2000 microns. Indeed, while shallower or deeper depths are possible with the current invention, channels of depths in this range seem to particularly effective at delivering cells, including functional cells, which can produce a beneficial response within the subject. In some embodiments, shallower channels are created, such as those measuring about 50 microns in depth. In other embodiments implementing deeper penetration, the channels can have a depth of about 2000 microns, even as deep as about 3000 microns. It should be appreciated, of course, that other measurements, such as falling between those stated, as well as measurements slightly larger or smaller than the stated numbers are also contemplated by the present invention, and that the above measurements are examples only and not meant to limit the scope of the invention. In at least one embodiment, in which the target site is skin, the channel(s) created in the skin tissue are at least deep enough to reach the lower layers of the epidermis. In some embodiments, the channel (s) have a depth sufficient to penetrate the dermis of the skin, or even subcutaneous tissues underlying the skin. In some embodiments, the channel (s) may extend as far as the bone marrow underlying the target site.

The depth and diameter of the channels may be specifically determined and created based upon the desired affect and/or ultimately desired delivery location of the bioactive agents. For example, the channel depth may be regulated to optimally deliver the bioactive agents at precisely the site at which they are needed such as locally, or as will be described, for the conveyance or migration of the bioactive agent (s) to a remote or distal site. Channel depth and diameter may also be optimized to treat broad areas at specific depths with diameters allowing for the best possible dosing of bioactive agents. This type of delivery cannot be accomplished by other means.

Each channel also includes an opening at the surface of the tissue of the target site, created by the laser light breaching or breaking the surface of the target tissue. The opening will therefore be defined by the laser light used to create the channel, and will therefore correspond to the dimensions of the channel. For instance, the opening of each channel may have the same width or diameter as the corresponding channel created in the tissue beneath the surface, at least initially. However, the channel begins to heal from the laser injury, the opening may be larger or smaller than the width/diameter of the interior channel, depending on the manner and degree of healing of the channel.

Depending on the desired therapy, a single channel or a preferably plurality of channels, such as disposed as a matrix of channels, can be created in the tissue with the laser. For instance, a scanning laser can create a matrix of 400 to 800 channels per square centimeter of tissue. Moreover, the channels can be arranged in the matrix in any orientation, such as parallel or perpendicular to each other, intersecting at angles, etc. Further, the laser can be adjusted to vary or alter the properties of the channels created. For example, in some embodiments, at least one of the channels created by the laser comprises coagulated edges based on the settings of the laser. It should be understood that each channel will have a corresponding opening at surface of the target tissue.

The laser light as applied to the tissue creates ablated channel(s) in the present invention so as to create an injury response in the subject, and specifically, a laser-generated injury response. This injury response may include an inflammatory response, including initiating a cytokine cascade whereby cytokines, immune cells, and other factors are recruited to the site, and other biological responses to the laser injury. These are natural, innate, or endogenous responses of the body of the subject. Moreover, this injury response may occur along the entire length of the channel (s), at any point of the tissue, which is damaged by the laser light. For instance, if laser-ablated channels are made that are at least 300 microns deep or more, the injury response occurs at least at 250 microns below the surface of the target tissue, and may occur continuously from the opening of the channel(s) to the deepest point of the channel(s), or at any point therebetween.

In some embodiments of the methods of the present invention, at least one bioactive agent is administered to the laser-treated site, specifically to the channel(s). For instance, the bioactive agent(s) may be applied topically or at the surface of the tissue at the target site, such as at the openings of the channel(s). The unique environment of the laser-created channels, and perhaps the biological response created thereby, serves to pull, recruit, or otherwise effectuate the migration of the administered bioactive agent(s) from the application site at the opening of the channel(s) down into the channel(s), and into the tissue surrounding the channel(s). Accordingly, the bioactive agent (s) need not be forced, pushed, manipulated, or actively moved into the interior of the channel. Rather, administration at the surface or opening of the channel is sufficient to allow the migration of the bioactive agent(s) into and through the channel(s). The administered bioactive agent(s) are similarly capable of migrating and/or being drawn, directed, or recruited through the walls of the channel and to distal sites separated or spaced apart from the target site of laser application and bioactive agent (s) administration.

The bioactive agent(s) administered to the channel(s) is as described and defined previously. In some embodiments, the bioactive agent(s) is administered with a pharmaceutically acceptable carrier or vehicle, such as an aqueous solution, saline solution, emulsion, cream, gel, or any other vehicle contemplated herein. The vehicle may be used to deliver the bioactive agent in real-time, or to release the bioactive agent in a controlled (i.e., time and/or dose dependent) manner into the channel.

In at least one embodiment, as noted previously, the bioactive agent(s) is at least one type of functional cell, which may be a stem cell or other type of cell. While stem cells treatments currently exist, known methods and treatments include applying hematopoietic hormones or growth hormones to spur the subject's body to release its own stem cells, or they a large number of stem cells are administered to the subject, such as by injection or intravenous (IV) injection. For instance, known methods require the application of 200 million stem cells or more to ensure some of the stem cells are viable upon injection and will reach their target destination and produce the desired result. In striking contrast, with the present invention far fewer cells need to be administered, and a greater or enhanced result is actually seen. For example, in at least one embodiment, the present method of delivery includes administering about 1 million cells or less as the bioactive agent(s) applied to the laser-ablated channels at the target site. This is ten times fewer cells than known stem cell treatments require. In some embodiments, approximately 750,000 cells are administered in the present method.

The functional cells or stem cells as delivered by the present invention can be used to reconstitute function and/or provide genes and proteins. Notably, the cells delivered by the various embodiments of the present invention, such as stem cells, remain viable and capable of dividing and producing proteins, even after migration into local tissue from the channel(s) or systemic migration to distal sites, at least in part because the subject's own endogenous physiological reaction to the laser ablation facilitates assimilation and utilization of the stem cells. Such cells can therefore be used to effectively deliver proteins, secrete factors, or other similar agents to a target site.

As mentioned above, the present invention may be used to systemically deliver one or more bioactive agents to a subject. That is, in at least one embodiment the method comprises applying a laser or laser light to a target site on the subject that is spaced apart from a distal site to be affected by the bioactive agent(s), creating at least one channel in the tissue at the target site, and administering the one or more bioactive agents to the target site at the channel(s) for migration into the channel(s) and recruitment to the distal site to achieve a biological response at the distal site. As noted previously, this can include the administration of about 1 million cells or less as the bioactive agent to the laser-created channel(s), and systemic delivery of the cells to the distal site will occur.

As used herein, a “distal site” is one that is separated or spaced apart by a distance from the target site where bioactive agent is initially applied. The distance or space separating the target site from the distal site is such that the two sites are not local to each other or co-localized. For instance, the distal site and target site may be located on different portions of the body of the subject, on different limbs, different tissues, or different bodily systems. Despite the distance between the distal and target sites, however, the distal site may be affected by bioactive agent(s) applied at the target site, as described previously. In some embodiments, the distal site is the site where a condition or disorder physically, phenotypically, or clinically manifests. For instance, and by way of illustrative examples only, applying laser light to create channels and apply bioactive agent at a target site on the skin of the arm of a subject may be used to treat liver disease or myocardial infarction, or to reconstitute immune function or vaccinate the subject.

As used herein, the terms “systemic delivery” and “systemically delivering” refer to the administration of a compound, factor, or other agent (i.e. bioactive agent(s)) such that it is recruited, dispersed, conveyed, directed, or otherwise migrates beyond just the site of application, such as throughout the body. The site of administration may occur at a single area, such as through contact with a single channel created by laser or through a matrix of channels created by a laser, or administration may occur at multiple areas, either on the same tissue or organ, or on multiple different tissues or organs at different sites throughout the body. Systemic delivery is accomplished once either: (1) one or more bioactive agents are detected at a distal site from the site of administration (i.e. the target site), or (2) a biological response is detected either systemically (i.e. at any point in the body of the subject other than the target site, including circulating such as in the vascular or lymph systems) or at a site distal to the site of administration.

The present invention can therefore be used to treat systemic conditions or diseases, or to treat conditions or diseases systemically. As used herein, “treating systemic conditions” refers to either treating a disorder that comprises systemic symptoms or treating a disorder that comprises localized symptoms at one or more sites distal to the area where the laser is applied. Examples of systemic disorders that the present method can be used to treat include, but are not limited to, distant organ damage such as myocardial infarction or chronic lung disease, reconstitution of immune function, reconstitution of hematopoietic function, treatment of vascular disorders such as peripheral artery disease, stroke, lymphedema, tissue or organ damage from injury including loss, as well as any disorder involving or characterized by the loss or lack of a gene, protein, or cell. The disorder treated by the methods of the present invention may be the result of an injury to a tissue or a cell, such as resulting from an environmental insult. The disorder treated can also be a sporadic (isolated, non-heritable event) or heritable genetic disorder. In at least one embodiment, the disorder treated is a burn wound on the skin or other tissue of a subject. The bioactive agent is administered to the site of the wound following channel creation by the laser. Additional examples of disorders or conditions which the present invention can be used to locally treat and/or local applications of the current method include, but are not limited to, skin disorders such as genetic-based skin disease, all forms of inflammatory skin disease, wound healing, scar reduction, tissue remodeling, skin regeneration, improved cosmesis of the skin (such as rhytids and solar elstosis), pigmentary disorders, all forms of hair loss or alopecia (including both medical and cosmetic), hair growth and regeneration, acute injury such as burns, revascularization, and generally any disorder involving or characterized by the loss or lack of a gene, protein, or cell.

For conditions involving or characterized by the loss or lack of a gene, protein, or cell, the present invention can be used for gene therapy and/or rescue and functional reconstitution or restoration. For instance, the bioactive agent(s) administered to the laser-created channel(s) include at least one corrective gene or gene product that is capable of providing a therapeutic benefit for a condition resulting from a corresponding aberrant gene or gene product. As used herein, “gene” is defined as a DNA sequence encoding RNA and/or protein upon transcription or translation, respectively, and may include introns, exons, promoter regions, enhancer regions, and combinations thereof. A “gene product” as used herein refers to RNA or protein resulting from transcription or translation of a gene, respectively. “Aberrant” indicates a departure from the naturally occurring or wild-type which often results in a condition, disorder, or disease. This can be the result of mutation, such as substitution, insertion, deletion, inversion, translocation, or chromosomal rearrangement, which may be a point mutation(s) or affect a region of nucleotides, leading to a missense mutation, nonsense mutation, null mutations, that can be hypomorphic, hypermorphic, dominant negative, loss of function, or gain of function mutations resulting in improper folding, misfolding, and/or non-functioning protein or RNA product. Such aberrant gene products can lead to apoptosis, necrosis, cellular defect, aberrant cellular growth and/or division, reduction or arrest of cellular growth, resulting in a disease, disorder, or condition such as cystic fibrosis or sickle cell anemia, by way of example only. The aberrant gene or gene product may be heritable or congenital, or be the result of damage from external and/or environmentally factors. “Corrective” indicates a sequence corresponding to the naturally occurring or wild-type that would yield a functional protein or RNA product for which no abnormal condition or disorder is associated. Administration of a corrective gene or gene product, such as in a cell(s) carrying and capable of expressing the corrective gene or gene product, therefore provides the appropriate correction to alleviate, decrease, reduce, abbreviate, slow, halt or reverse a condition caused by an aberrant gene or gene product. Accordingly, “therapeutic relief” as used herein means a physical, phenotypic or clinical expression of the correction of an aberrant gene or gene product, such as restoring or reconstituting function, and may include correction at the nucleic acid, protein, cellular, tissue, organ, system, and/or organism level. Moreover, correction may be in an upward or downward direction. For instance, even overexpression or overabundance of a particular gene, allele, protein, peptide, or cell can be corrected with the present invention, operating with inhibitory or blocking genes or gene products. Corrective gene or gene products may also include tags, markers, biomarkers, or other ways of following and/or identifying the corrective gene or gene product, so as to verify its presence at a particular location. Accordingly, the corrective gene or gene product may be transgenic and/or chimeric.

Alternatively, the cell carrying the corrective gene or gene product may express some such marker for identification purposes separate from the corrective gene or gene product itself. Further a mixture of stem cells and cells bearing the corrective gene or gene product may be administered with the present invention for enhanced gene therapy or functional restoration.

In embodiments involving the treatment of a systemic condition, channels can be made and the bioactive agent may be administered directly at the organ, or preferably, at a separate delivery target site, such as on the skin, spaced apart from the site affected by the condition to be treated. Channel(s) can be created as described previously at a predetermined depth at the target site so as to cause a reaction in the body that promotes systemic migration and/or conveyance of applied bioactive agents so they can be effectively recruited to the remote organ in need at the distal site. This may be sufficient to create an injury response, such as a laser-generated injury response, at least 250 microns below the surface or more depending on the predetermined depth of the channel(s). The channels may also be sufficiently deep to penetrate deep layers of the epidermis, or to at least penetrate the dermis in some embodiments. Moreover, it may be ideal to define channels that extend into a region of high capillary activity that will facilitate conveyance of the bioactive agents through the bloodstream. In some cases channel depth and diameter may need to be optimized to deliver materials to areas where delivered agents, such as cells, can establish themselves for later systemic distribution. Examples of how this may occur include the delivery of hematopoietic cells to the sub capsular space of a lymph node or spleen, or the delivery of mesenchymal cells or fibrocytes to the sub epidermal/dermal space in the skin, although systemic delivery is certainly not limited to these examples.

In addition, administration of certain substances at one location may affect an organism systemic cascade in a positive way. Further, the depth of the channel can be used to deliver bioactive agent such as stem cells or therapeutics to a desired level, for example, to treat infection, carcinoma, tumor, or replacement of organs in diseased tissue.

As mentioned previously, with the present invention, once the bioactive agent is introduced into the subject by means of the channels at the target site, the subject's physiology recognizes the presence of the bioactive agent and recruits it/them to the necessary organ or remote site of injury as though the bioactive agent was naturally occurring or produced. As a result, a minimally invasive and pain free delivery of the bioactive agent can be achieved while still producing a very targeted and tolerable delivery. This is the first time transdermal administration has been shown to be capable and effective for systemic delivery. Previously, only open or invasive procedures, or injection, could provide systemic delivery. Moreover, viable and functional cells capable of cellular division and protein expression can be delivered to systemic and/or distal sites using the present invention and retain their viability and functionality at the systemic/distal site, as shown in the Figures and Examples. T his, too, has not been shown heretofore.

Additionally, it is also noted that because of the systemic disbursement that can be achieved using the present system and method, the present invention includes a method and/or use for vaccination and treating immune tolerances. In such vaccination embodiments of the present invention, the bioactive agent is a vaccine for a particular antigen, which may be any predetermined antigen that the subject may be in need of vaccination against.

Although the bioactive agent(s) will be delivered systemically using the method described above, a distal site may optionally be subjected to laser light application, to create at least one, or a plurality or matrix of, channels as described previously at the distal site. This channel(s) created at the distal site will draw or direct the bioactive agent(s) more efficiently to the distal site for beneficial action and biological response. Indeed, even if the bioactive agent is delivered by some other route, such as by injection or IV, and is already circulating throughout the subject, creation of laser-generated channel(s) at a particular identified site to be affected will direct the circulating bioactive agent to that identified site. Regardless of how the bioactive agent is initially introduced to the subject, the laser-ablated channels created at the distal site, or a particularly identified site for treatment or action, are sufficient to initiate an injury response as previously described, which may include a laser-generated injury response. Accordingly, the present invention may also be used to direct at least one bioactive agent, such as a functional cell, to a particular site.

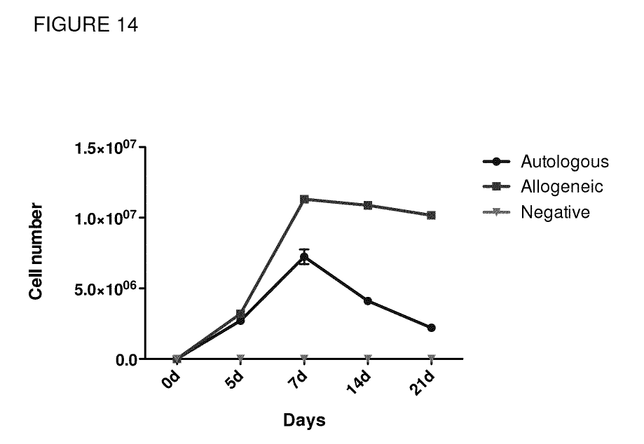

As noted previously, the present invention provides a number of benefits and/or positive reactions in the body. One of these positive reactions is the increased circulation of the subject's own endogenous stem cells following treatment, application, or use of the present method of bioactive agent delivery, as shown in FIG. 14. Specifically, applying laser light to an identified target site on a subject to create at least one channel at the target site, and administering at least one bioactive agent, such as a functional cell, to these laser-ablated channel(s) as described previously results in the stimulation of the production of the subject's endogenous stem cells in circulation within the subject. This may be the result of increased stem cell production, but at the very least is a result of increased release of stem cells into circulation. Accordingly, the present invention may be used to boost or enhance the subject's own healing abilities and mechanisms. As before, the target site where the channel(s) are created may be a site on the skin of the subject, and the channel(s) created therein may be at least deep enough to create a laser-generated injury response and/or penetrate the dermal layer of skin. Also as before, the bioactive agent used in these embodiments may be at least one functional cell, such as at least one type of stem cell, for example mesenchymal stem cells. Also, approximately 1 million cells or fewer may be administered to the channel(s) to achieve this stimulatory effect. In at least one embodiment, the bioactive agent administered to the channel(s), such as the type of functional cell, corresponds to the type of cell whose endogenous circulation is stimulated by the present invention.

Accordingly, the stimulation in production of endogenous stem cells is in response to the laser-generated injury, which results in a greater number of endogenous stem cells circulating in the subject than ordinarily circulate, or would be circulating in response to other non-laser generated injuries as described herein. Moreover, in at least one embodiment the stimulated endogenous stem cell production is the result of an increase in the release of stem cells from tissue, such as bone marrow, into circulation, from such laser-generated injury. In another embodiment, the stimulated endogenous stem cell production is due to an increase in growth or replication of these stem cells once in circulation. In view of these aspects, the present invention may therefore also be used to harvest stem cells in a more efficient, less intrusive, less painful, and less expensive way than current harvesting methods, which involve bone marrow extraction. A simple blood sample obtained a few days after application of the present method as described herein would provide plentiful stem cells, which may be re-administered to the subject, such as at a later time, stored for future use, cultured, or used in other patients for other treatments.

In at least another embodiment, the present invention is directed to a method of delivering one or more bioactive agents to a subject in need thereof using a tissue explant. Such method comprises applying laser light to a tissue explant, creating at least one channel of a predetermined depth and/or diameter in the tissue explants. Application of such laser light is as previously described, and the channel(s) created thereby are also as previously described. The method further includes administering one or more bioactive agent to the tissue explants, such as at the opening or surface of the channel(s), to obtain a seeded tissue explant, and implanting the seeded tissue explant into the subject. The bioactive agent applied to the tissue explant may be any of the possibilities discussed previously, including functional cells. Upon implantation, the bioactive agent(s) seeded within the tissue explant migrate out of the explant and at least into the surrounding tissue of the implant recipient. It may also penetrate further, as systemic migration of the bioactive agent(s) from the explant to distal sites for action there is also possible and contemplated herein. For example, the seeded cells or bioactive agent(s) may depress an innate immune response to the delivered material and allow the cells or bioactive agent(s) to exit the explant and be distributed in order to deliver a therapeutic effect. Accordingly, such use of a tissue explant may be used to deliver bioactive agent to a distal site via systemic delivery, such as in the treatment of systemic conditions or vaccination, and may even be used as a gene therapy vehicle or for functional restoration, as outlined previously.

As used herein, a “tissue explant” refers to a section of tissue, which may be synthetic or organic in nature. For instance, in at least one embodiment the tissue explant is synthetically formed from a biologically inert material that does not cause an immune response or graft-versus-host disease. Possible examples include, but are not limited to, polyethylene glycol (PEG), which may be in hydrogel or other form, biologically inert acrylics or polymers such as polymethyl methacrylate (PMMA), silicone, and others. Alternatively, the tissue explant may be of biologic origin, such as taken from a living organism, as in the case of being excised or taken from the body of an animal. The donor animal may be the subject in which the explant will later be implanted after being seeded with bioactive agent, in which case the explant is autologous. In other embodiments, the donor is a different animal, such as of the same species (for allogenic tissue explants) or a different species. Moreover, the tissue explant may be cultured or stored ex vivo until such time as is desired to be seeded and used in the present invention.

In some embodiments, the seeded tissue explant is implanted into a tissue of the subject. Implantation may occur at any appropriate depth, and in at least one embodiment is implanted to a predetermined depth so as to enable systemic movement of the bioactive agent(s) from the seeded explant to distal sites separated or spaced apart from the implantation site. The explant may be attached to skin tissue, such as at the exterior surface of the epidermis. The tissue explant may also be implanted subcutaneously. In other embodiments, the seeded tissue explant is implanted into an organ of the subject, such as through surgery. It is also contemplated that the seeded tissue explant could be implanted within the interstitial and third spaces of the subject's body.

In addition to the previously defined methods and uses, the present invention is further directed to a system for delivering one or more bioactive agents to a tissue. Such system comprises a laser instrument capable of producing at least one beam of laser light that can create at least one channel of predetermined depth in tissue at a target site on a subject and having an opening at the surface thereof, as described above. The laser instrument is preferably adjustably configured to produce an array of channels of a predefined depth and diameter, said adjustability in one preferred embodiment being achieved by varying the power output, desired channel density and/or duration of the laser's operation. Such lasers and the channels they are capable of producing have been described previously herein.

In addition to the laser, the system further includes one or more bioactive agents that are disposable at the opening of the channel(s) and capable of migrating into and through the channel(s) as well as systemic migration to distal sites for beneficial effect, as described previously. As before, the bioactive agent(s) may be at least one type of functional cell, including stem cells. The system may therefore include at least 1 million of such functional cells or fewer to be applied to the channel(s). Also as before, the bioactive agent in some embodiments may be a cell, protein, peptide, peptide fragment, nucleic acid, nucleotide fragment, gene, pharmaceutical compound, therapeutic compound, medicament, small molecule, aptamer, and combinations thereof. The system is structured to deliver the bioactive agent(s) locally or systemically, as described above. The system may also further comprise a tissue explant, as described previously, which in some embodiments comprises the at least one bioactive agent.

The present invention is also directed to a kit of parts comprising one or more bioactive agents and other reagents needed to perform the method(s) of the present invention, as well as instructions for use of the same, including in the application of the bioactive agent(s) to laser-ablated channels created in a target tissue. The one or more bioactive agents and reagents can be included in one or more compositions, and each bioactive agent and reagent can be in a composition in combination with a suitable vehicle, or can be present independently. The bioactive agent present in the kit may be any of the previously described bioactive agents, available in any concentration or in any acceptable and suitable carrier or solvent. Moreover, the bioactive agent may further include a preserving or stabilizing agent to prolong the useful life of the bioactive agent in the kit, to enable storage and later use. In some embodiments, the kit of parts also includes labeling markers for the bioactive agent, reference standards, and additional components that would be identifiable by those skilled in the art upon reading the present disclosure. The labeling marker comprises expression plasmids, vectors, viruses, unique nucleotide or peptide sequences, dyes, fluorescent markers, and the like, which allow for tracking the bioactive agent by molecular or non-molecular (e.g. MRI or X-ray) techniques.

In some embodiments, the kit further includes a laser, which may be mobile or hand-held such as for field use, and additional instructions for using a laser device in combination with the components of the kit of parts. As before, the laser may be ablative or a fractional ablative laser, and is capable of creating channel(s) in target tissue as described previously. Accordingly, accompanying instructions may include programming settings for the laser or software that operates the laser, as well as instructions on channel placement and creation. Moreover, these instructions may vary depending on the particular application or desired outcome for which the kit and bioactive agent(s) is being used.

In additional embodiments, the kit may also include a tissue explant, which may be pre-seeded with bioactive agent(s) or not, as well as instructions for use or implantation of the tissue explant to deliver the bioactive agent, as described above. In some embodiments in which the tissue explant is provided in the kit in a non pre-seeded form, the kit may also include instructions on creating laser-ablated channel(s) in the tissue explants and/or administration of the bioactive agent(s) to the channel(s) created therein to form a seeded explant.

The present invention also admits of various other beneficial uses. For instance, the present invention may be used to provide treatment or beneficial response at a local site at which the laser light is applied and channels are created. In at least one embodiment the present invention is directed to a laser-assisted method of treating skin disorders in a subject in need thereof, whereby the treatment includes applying a laser to the skin of the subject, creating at least one channel in the skin to a desired depth and which may vary depending on the skin disorder being treated, and administering one or more bioactive agents to the skin at the channel(s) created at the target local site. Examples of skin disorders treatable by this method have been provided previously.

In some embodiments, the skin disorder is a wound or skin infection. As is understood by those skilled in the art, a biofilm is a collection of microorganisms that are encased in their own extracellular matrix, which may form over or within a wound or skin. This biofilm has been shown to block or hinder delivery of medicaments or other reagents that could be used to kill the organisms, such as bacteria. The embodiments of the present invention are applicable to such conditions in that the laser is capable of piercing, penetrating, or breaking up the biofilm to allow delivery of bioactive agent(s) therethrough beyond the biofilm. Specifically, the laser energy is capable of penetrating the biofilm, thereby disrupting the barrier properties of the biofilm. This disruption can be capitalized upon, permitting administered bioactive agent(s) to access the tissue and organisms underneath the biofilm, which is ordinarily resistant to penetration. In accordance with another embodiment of the present invention, a method of disrupting a biofilm on a subject comprises applying laser to a local site on the subject. In some embodiments, the local site may be on the skin of a subject. Furthermore, the skin of the subject may comprise a disorder, such as, but not limited to, a wound or skin infection. The current embodiment may also be used in conjunction with other embodiments of the present invention to disrupt a biofilm in concert with delivering one or more bioactive agents to a local site, such as antibacterial or antimicrobial agents and/or cells expressing or capable of expressing antimicrobial agents or compounds.

In some embodiments, at least one channel is created through the biofilm. In other embodiments, channel(s) are not necessarily formed, so long as the biofilm barrier is sufficiently disrupted to allow bioactive agents therethrough. Accordingly, the present method provides an effective way of delivering bioactive agents, which may be antibiotics, antimicrobial peptides, anti-infective molecules, antibacterial agents or compounds, or any antimicrobial products, to a wound or skin lesion, such as but not limited to atopic dermatitis and acne, despite the presence of a biofilm that would otherwise obstruct such treatment. As used herein, “antibacterial” means capable of destroying, killing, reducing the effectiveness, and/or inhibiting the growth of a bacteria. “Antimicrobial” as used herein means capable of destroying, killing, reducing the effectiveness, and/or inhibiting the growth of any infective microorganism, which may include bacteria, protozoa, fungus, virus, mycoplasm, or other similar organisms that cause infection. Moreover, in addition to the benefits associated with facilitating the passage of the bioactive agent beyond the biofilm, it is also recognized that in some cases it may simply be preferred to disrupt the biofilm itself to promote natural healing or help with the penetration of any anti-infective agent or medicament after it has already been applied to the site. For example, in at least one embodiment of the present invention, a laser is applied to an area affected or covered by a biofilm, bioactive agent(s) such as antibiotics or antimicrobial products are applied, and a second round of laser treatment is applied to the affected area. This additional laser treatment following administration of topical agents facilitates the penetration of such agents deeper into the skin and the killing of organisms responsible for, contributing to, and/or comprising the biofilm.

The present invention is also directed to a method of growing hair. In some embodiments, the method can be used to regrow hair, such as in the case of hair loss, baldness, alopecia, or other condition in which hair has stopped growing. In other embodiments, the present method may be used to grow nascent hair, such as hair that has not grown before. In any event, the method of growing hair comprises applying a laser to a target local site on the subject. The target local site is any site on a subject where hair growth is desired. In at least one embodiment, the laser applied thereto is a fractional laser, and may be ablative or non-ablative. The method of growing hair further comprises creating at least one channel of a predetermined depth in the tissue of the target local site, such as by application of the laser to the site. The present method further comprises applying at least one bioactive agent to the target local site. In at least one embodiment, the bioactive agent is cells. In some embodiments, the bioactive agent is stem cells, progenitor cells, or other multipotent cells. The cells may be a homogenous population, such as comprising all stem cells, or may be a heterogenous mixture of cells. In one embodiment, the cells applied to the target local site for hair regrowth are lineage negative (lin(−)) cells. In another embodiment, the cells applied are a mixture of mature cells and multipotent cells. Further, the cells may originate in any tissue or system of the body. For example, in at least one embodiment, the cells are bone marrow cells.

Without further elaboration, it is believed that one skilled in the art can, using the preceding description, utilize the present invention to its fullest extent. The following examples are offered by way of illustration, not by way of limitation. While specific examples have been provided, the above description is illustrative and not restrictive. Anyone or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By citation of various references in this document, Applicant does not admit any particular reference is “prior art” to their invention.

### EXAMPLES

The methods and compositions herein described and the related kits are further illustrated in the following examples, which are provided by way of illustration and are not intended to be limiting. For instance, although bone marrow stem cells including hematopoietic and mesenchymal stem cells are used in the following Examples, many different types of cells have been used by the Applicants in testing the present invention; stem cells are presented herein as merely one example, and the present invention should not be limited thereto. It will also be appreciated that variations in proportions and alternatives in elements of the components shown will be apparent to those skilled in the art and are within the scope of embodiments of the present invention. Theoretical aspects are presented with the understanding that Applicants do not seek to be bound by the theory presented.

The following material and methods were used in the methods and compositions exemplified herein.

### Production and viral transduction of porcine allogeneic mesenchymal stem cells (MSC)

Bone marrow was withdrawn from a donor pig and mesenchymal stem cells (MSC) were established by routine methods known by those skilled in the art. Donor MSC's were then transduced with lentivirus containing expression vector(s) comprising a yellow fluorescent protein (YFP) nucleic acid sequence resulting in protein expression of YFP exclusively in the nucleus of transduced MSC cells. The transduction vector(s) also contained a non-expressed sequence unique to the vector for later use in molecular tracking of the transduced MSC.

### Laser Information

A fractional ablative Erbium-YAG laser (Sciton Profractional XC having spot size 430 microns, depth range 20-1500 microns, treatment densities 11% or 22%, and spot size 1-8) was used in many of the experiments. This Erbium-YAG (“Er:YAG”) laser is a tunable ablation laser and has coagulation options with specific tunable options. It also has the ability to change coagulation on three levels 0, 1, 2, 3. Experiments using the Er:YAG laser produced channels having depths of 35-1500 microns. Some experiments utilized a second device, a Lumenis Ultrapulse laser, using a 10,600 nm wavelength at 240 watts, 225 mJ of energy. The Lumenis Ultrapulse (CO₂) laser was used to create channels ranging in depth from 150 microns-1500 microns. The lasers used in the experiments described herein are capable of creating channels of many depths, including as deep at 2000-3000 microns. Specialized software was also employed with each laser.

### Full Thickness Wound Model

Full thickness wounds, which is understood to be tissue destruction extending through the second layer of skin (dermis) to involve subcutaneous tissue underneath, were created on the paravertebral and thoracic area of pigs with a 10 mm circular biopsy punch. Immediately after wounding, the wounded area was treated with one of the following fractional lasers (CO₂, 10,600 nm or Er:YAG, 2940 nm) to create microscopic (120-300 micron wide) vertical holes of ablated tissue to deliver the MSCs. Wounds and surrounding normal skin were then covered with an occlusive polyurethane film dressing (Tegaderm; 3M, St. Paul, Minn.). The MSCs (500 μl, approximately 750,000 cells) were injected through the polyurethane film dressing with a sterile syringe to allow access to laser channels. A secondary polyurethane film dressing was used to keep the MSCs in place. Control treatment groups included laser sites with saline and occlusive dressing only. Occlusive dressings were changed on days 7. After 14 days, wounds were covered with non-adherent gauze and these were again changed on days 21, 28 and 35. Three biopsies were taken from each treatment group on days 5, 7, 14 and 35 for histological analysis (see results in Microscopy section below). Burn Model Sixty (60) second degree burn wounds were created on the paravertebral and thoracic area using five specially designed cylindrical brass rods weighing 358 g each that were heated in a boiling water bath to 100° C. A rod was removed from the water bath and wiped dry before it was applied to the skin surface to prevent water droplets from creating a steam burn on the skin. The brass rod was held at a vertical position on the skin for six seconds, with all pressure supplied by gravity, to make a burn wound 8.5 mm diameter×0.8 mm deep (second-degree burn). Immediately after burning, the roof of the burn blister, which is characteristic of a second degree burn, was removed with a sterile spatula. Immediately after burning, the wounded area was treated with one of the following fractional lasers (CO₂, 10,600 nm or Er:YAG, 2940 nm) to create microscopic (120-300 micron wide) vertical holes of ablated tissue to deliver the MSCs. Wounds and surrounding normal skin were then covered with an occlusive polyurethane film dressing (Tegaderm; 3M, St. Paul, Minn.). The MSCs (500 μl, approximately 750,000 cells) were injected through the polyurethane film dressing with a sterile syringe to allow access to laser channels. A secondary polyurethane film dressing was used to keep the MSCs in place. Control treatment groups included laser sites with saline and occlusive dressing only. Occlusive dressings were changed on days 7. After 14 days, wounds were covered with non-adherent gauze and these were again changed on days 21, 28 and 35. Three biopsies were taken from each treatment group on days 5, 7, 14 and 35 for histological analysis (see results in Microscopy section below).

### Biofilm Model

Rectangular wounds measuring (10 mm×7 mm×0.5 mm) were made on the paravertebral and thoracic area (porcine) with a specialized electrokeratome fitted with a 7 mm blade. Wounds were inoculated with 250 μL of a 106 CFU/ml suspension and covered individually with a polyurethane film dressing to allow for biofilm formation. After 24 hours, the dressings were removed and wounds were randomly assigned to one of the following treatment groups: 1) untreated control, 2) ErYag, 3) ErYag plus gentamycin or 3) gentamycin alone. After treatment the wounds were covered with a new polyurethane film dressing and after 24 hours wounds were recovered for MRSA counts using a well published scrub technique. Oxicillin resistance screening agar (ORSAB) was used to isolate MRSA from the wounds. All plates were incubated aerobically overnight (16-24 hours) at 37° C., after which the number of viable colonies will be counted.

### Microscopy

At time points between 5 and 35 days post treatment, treated wounds were harvested and tissue divided for analysis. Analysis included histologic evaluation by routine light microscopy (formalin fixed paraffin embedded) and fluorescent imaging (by frozen section). Nucleic acid extraction was also performed from tissue and blood samples obtained.

DAPI staining was performed by placing a drop of VECTASHIELD® Mounting Media containing DAPI on the slide after which the slide was coverslipped.

Fluorescent and DIC microscopy was performed using an inverted 1×81 Olympus microscope (Olympus America, Center Valley Pa.) and ORCA-AG Hamamatsu digital camera (Hamamatsu Photonics K.K., Hamamatsu City, Shizuoka Pref. Japan).

Light microscopy was performed with an Olympus BX51 upright microscope and Olympus DP-72 camera (Olympus America, Center Valley Pa.).

### Example 1

### Laser-Assisted Delivery of Viable Cells to the Skin

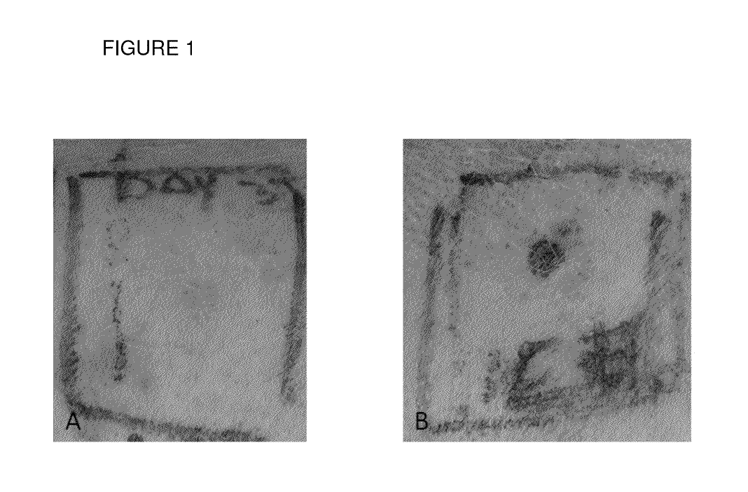

Porcine dorsal skin was inflicted with a second-degree burn injury using a brass rod heated in a water bath as described above, then treated with either (a) fractional laser ablation and allogenic porcine mesenchymal stem cells (MSC) transduced as described, or (b) covered with polyurethane film, wherein no cells were delivered, to act as a control. FIG. 1 shows that 35 days after treatment, the burn wound treated with laser ablation and subsequent delivery of MSC (panel A) resulted in significant cutaneous healing and recovery, with less inflammation, crusting, local edema, and reduced scarring compared with the control, non-treated burn wound (panel B).

### Example 2

### Cells Delivered by Laser-Assisted Delivery Migrate to Local Tissue in Full Thickness Wound Model

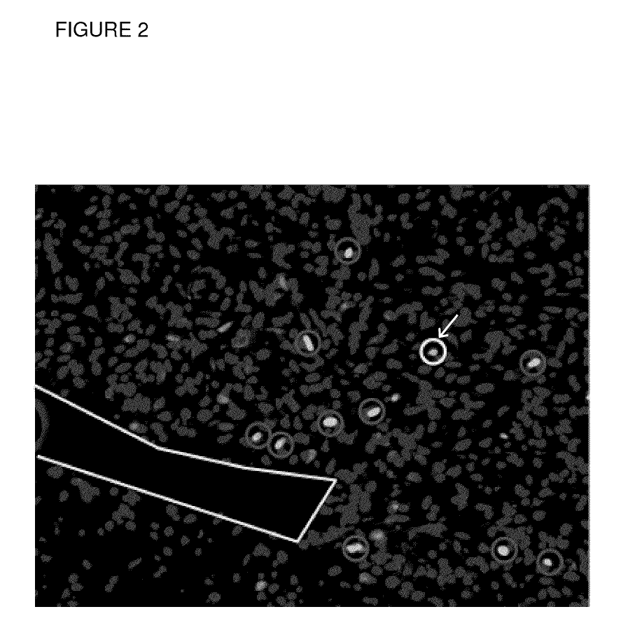

Since the transduced MSC's used in these studies expressed a fluorescent marker in their nuclei, they could potentially be tracked by direct immunofluorescence to analyze the migration of cells. To analyze this, full thickness wounds (skin lesions) were created using a disposable 10 mm punch biopsy instrument, and were treated with laser ablation and subsequent application of MSC as described above. Biopsy samples were obtained at specific time points. The specimen shown in FIG. 2 was taken 5 days after laser treatment and application of MSC's. Frozen sections were prepared, stained with DAPI and then visualized with fluorescence microscopy at 200× magnification to determine location of transduced MSC expressing YFP. As shown in FIG. 2, the transduced cells (indicated by circles) migrate from the channel created by laser ablation (outlined by rectangular lines) to the locally surrounding or adjacent tissue (field of DAPI-stained nuclei) upon application to the laser-ablated channel. The circle indicated by the arrow highlights an auto fluorescent area that is common in wounded tissue. This indicates the present method, systems and kits can be used to locally and/or directly treat disorders and injury, such as on the skin or directly to organs in the case of open surgery.

### Example 3

### Cells Delivered by Laser-Assisted Delivery Migrate to Local Tissue in a Burn Model

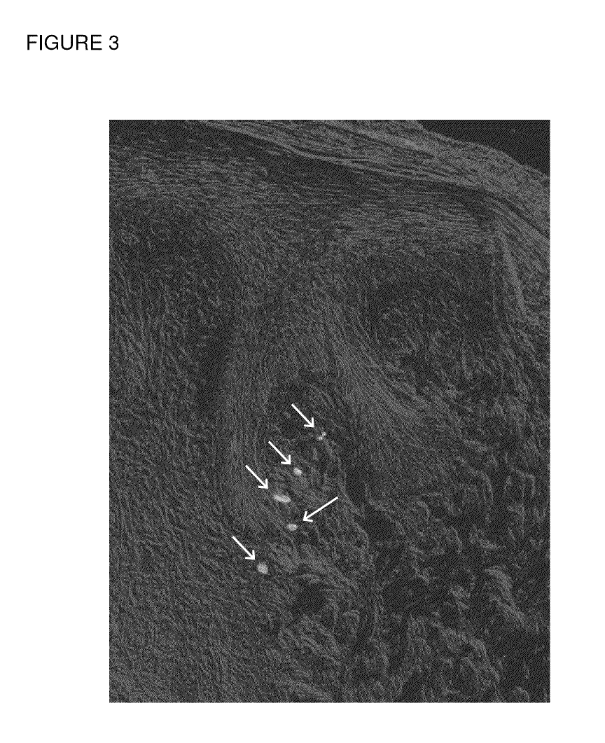

Porcine skin was injured by the burn wound protocol as described above and treated with fractional laser ablation and subsequent application of YFP-transduced MSC. After seven (7) days post-treatment, a sample of the treated tissue was obtained and processed by frozen sectioning. The sections were examined for transduced MSC's by fluorescent microscopy with background structures highlighted by false color differential interference contrast (DIC) image overlay (all done at 100× magnification). As shown in FIG. 3, the MSC's (indicated by arrows) are arranged in linear fashion down the ablated channel created by the laser. Accordingly, cells delivered by the present methods are also delivered to the skin in burn models.

### Example 4

### Cells Delivered by Laser-Assisted Delivery are Present in the Crust

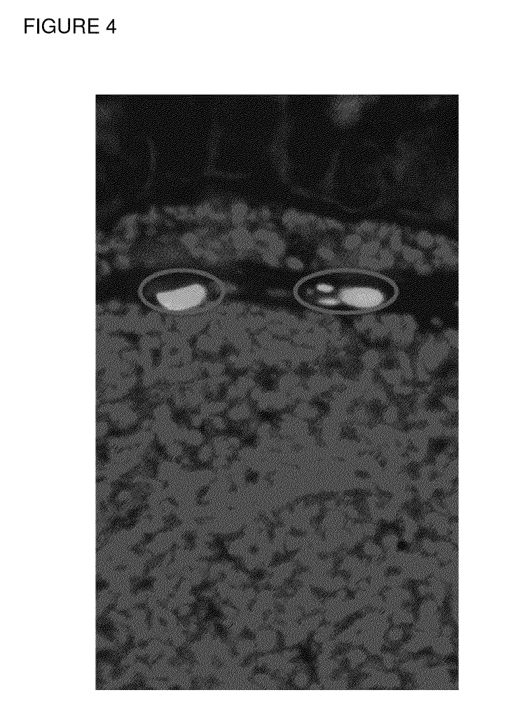

Crusting over the burn wounds occurs for several days due to the elimination of devitalized skin. To test whether cells delivered by laser-assisted delivery are present in the crust, porcine skin, both epidermal and dermal, was injured by the burn wound protocol as described above, then treated with fractional laser ablation and subsequent application of YFP-transduced MSC. After seven (7) days post-treatment, a sample of the treated tissue was obtained and stained with DAPI to identify nuclei. The crust over the burn site was then visualized using fluorescence microscopy at 400× magnification. As shown in FIG. 4, the YFP-expressing MSC (lighter colored cells that are circled) are present in the crust over the burn site. Moreover, while the nuclei of many of the surrounding cells in the crust are small and degenerating, the MSC remain viable in the crust.

MSC's are known to contribute healing effects by secreted agents. The successful delivery of viable MSC's to the crust would potentially allow for the delivery of any secreted agent produced by these cells, whether native to the cell or manufactured by a transgene.

### Example 5

### Improved Healing from Laser-Assisted Delivery of MSC in Full Thickness Wound Model

To test the ability of MSC's to contribute to the healing of a wound, porcine skin tissue was injured by the full thickness wound protocol as described above and then treated with fractional laser ablation and subsequent application of YFP-transduced MSC.

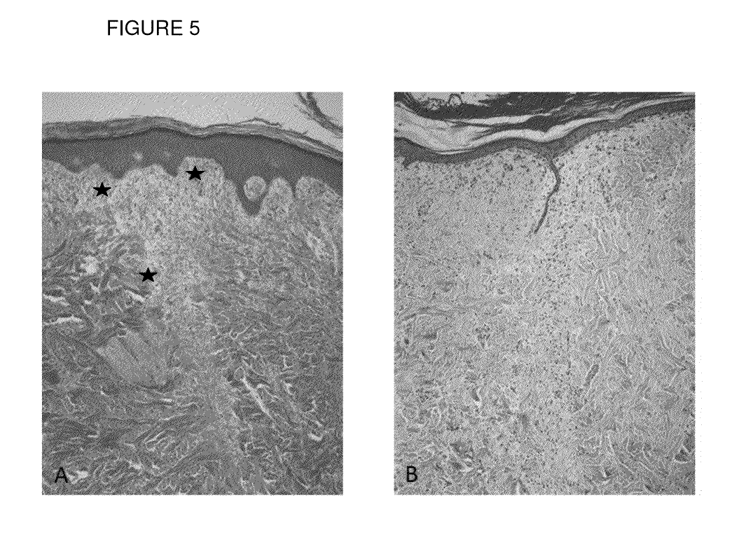

Control tissue was similarly injured with the full thickness wound protocol and treated with fractional laser ablation, followed by application of saline. Fourteen (14) days after treatment, tissues biopsies were obtained and analyzed for signs of healing, both clinically by implementing digital photography, and histologically by taking measurements of epithelialization, epithelial thickness and estimate of inflammatory infiltrate. As shown in FIG. 5, the MSC-treated dermis (panel A) shows improved wound healing, as shown by a fetal collagen-like arrangement of the cells in the tissue (indicated by stars), and thicker epidermis compared to the control cells (panel B).

### Example 6

### Improved Healing from Laser-Assisted Delivery of MSC in Burn Model

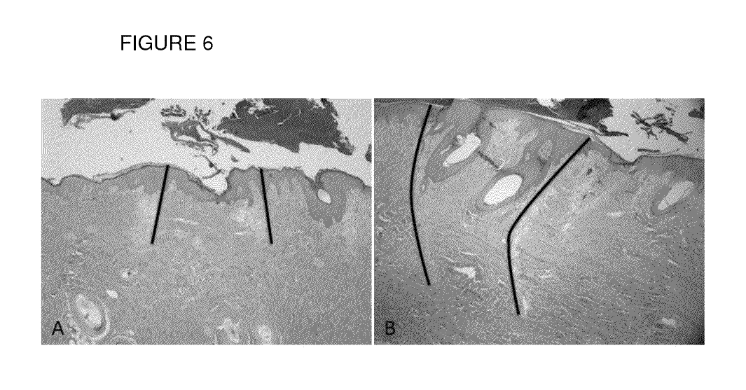

Likewise, the ability of MSC's to contribute to the healing of a burn wound was tested. Porcine epidermal and dermal tissue was injured by the burn wound protocol as described above and then treated with fractional laser ablation and subsequent application of YFP-transduced MSC. Control tissue was similarly injured with the burn wound protocol and treated with fractional laser ablation, followed by application of saline. Seven (7) days after treatment, tissue biopsies were obtained and analyzed for signs of healing. As shown in FIG. 6, the MSC-treated tissue in panel A shows that much of the channel (indicated by black lines) has been healed, having an average depth of 0.8 millimeters, as compared to the control in panel B, in which the channel depth has an average depth of 2.2 millimeters (highlighted by black lines). Therefore, improved healing was observed in both the full thickness wound model and the burn model.

### Example 7

### Transduced MSC Delivered by Laser are Present in Bone Marrow

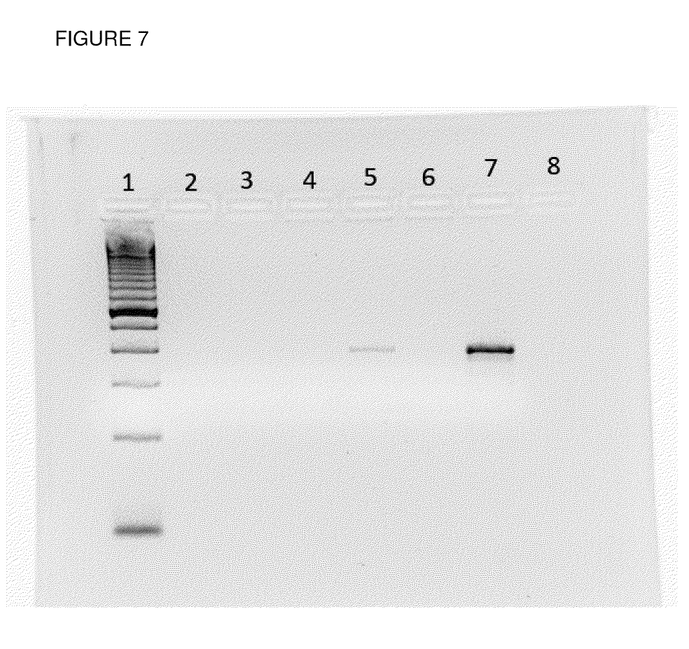

To test for the ability of cells administered by the laser-assisted delivery technique to be delivered to distant sites, porcine skin was treated with fractional laser ablation and transduced MSC were applied topically with an occlusive dressing or chamber, as described above. After three (3) weeks, a bone marrow sample was taken from the treated pig, as was bone marrow from two non-treated pigs as a negative control. The genomic DNA from each sample was isolated, and the lentivirus specific gene was amplified by PCR. The results were run on an agarose gel, stained with ethidium bromide and visualized under ultraviolet light. As shown in FIG. 7, lane 1 shows a 100 base pair (bp) ladder to indicate the size; lane 2 provides a non-template control template; lane 3 is control porcine genomic DNA template from a first untreated pig; lane 4 is control porcine genomic DNA template from a second untreated pig; lane 5 is DNA template from the pig treated with laser ablation and transduced MSC; lane 6 is control human genomic DNA template; lane 7 is a positive control of the lentivirus plasmid DNA, that was transduced into the MSC, as template; lane 8 is empty. As is readily apparent from the band at approximately 384 bp in Lane 5, the bone marrow of the treated pig shows the presence of transduced MSC, indicating that cells migrated to the bone marrow from the laser-ablated channel at the skin. Therefore, these results suggest that cells administered using the techniques described can be delivered to distant sites and can persist. The persistence of these allogeneic cells is somewhat longer than expected and could indicate a mechanism of immune tolerance for cells delivered by this method since the route of administration is unique in these studies and may have several immune based advantages.

### Example 8

### MSC's delivered by laser-ablated human skin explants migrate to the bone marrow

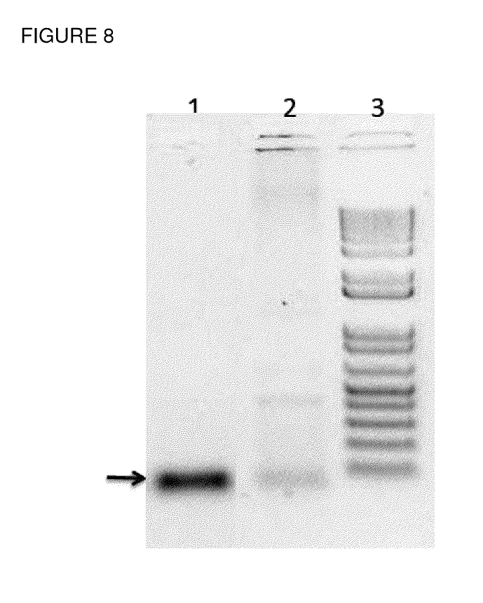

Human mesenchymal stem cells (MSC) were transduced with the same lentivirus as described above to confer nuclear-specific expression of YFP. Human skin explants were obtained from human subjects under an IRB approved protocol, treated with laser ablation, and seeded ex vivo with the transduced human MSC's. The seeded explants were then implanted into immune-compromised NOD/SCID mice. In the weeks following implantation, no significant inflammatory or foreign body reaction was detected in the recipient mice. After four (4) weeks, the mice were sacrificed and their bone marrow was collected. Genomic DNA from each sample was isolated, and the lentivirus specific gene was amplified by PCR. The results were run on an agarose gel, stained with ethidium bromide and visualized under ultraviolet light. As shown in FIG. 8, lane 1 is the lentivirus positive control as template; lane 2 is murine DNA template from the mouse receiving implant seeded with transduced human MSC's; lane 3 is a 1 kilobase (Kb) ladder to show size. Accordingly, the bone marrow of the recipient mice show the presence of the lentivirus, indicating the transduced MSC's migrated systemically from the explant in which they were seeded to the bone marrow of the host. Therefore, cells seeded within a matrix can be delivered systemically to distant organs using the present methods, systems, and kits.

Since no significant inflammatory or foreign body reaction was detected in these mice in the weeks following implantation, it is indicative of the seeded MSC's in the explants suppressing the reaction. Applicants have previously observed inflammatory or foreign body reactions in other such experiments where mice were implanted with explants without seeding with MSC's. Furthermore, this study shows that seeded cells within a matrix using the laser assisted delivery technique could be delivered to distant organs. This explant/matrix technology represents a versatile element of this platform technology.

### Example 9

### Cells Delivered by Laser Remain Functionally Intact

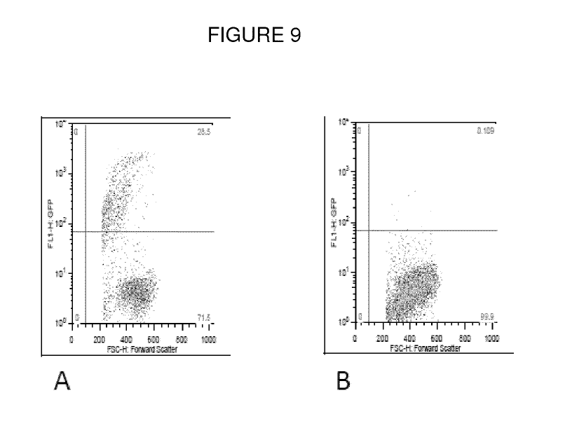

It was hypothesized that cells could be delivered through skin treated with fractional ablative laser and that the delivered cells could remain functionally intact. To test this hypothesis, green fluorescent protein (GFP) positive bone marrow cells were derived from donor GFP expressing transgenic mice (C57BL/6-Tg (UBC-GFP) 30Scha/J) to be delivered to non-GFP expressing recipient mice. Immune deficient NOD/SCID recipient mice were irradiated (or not irradiated, in the case of the control mice) to create space in the bone marrow compartment, and were subsequently treated with fractional laser ablation at the skin. GFP expressing bone marrow cells were delivered by securing the seeded plastic chamber to the laser-ablated skin of the mice using adhesive. Three (3) weeks after a single treatment, blood samples were taken from the recipient mice. FIG. 9 shows fluorescence-activated cell sorting (FACS) analysis of the blood samples. Panel A shows that mice receiving radiation, laser treatment and cell delivery show 28.5% of the circulating nucleated cells express GFP, confirming chimerism of the bone marrow. Panel B shows that control mice that did not receive radiation did not have circulating fluorescent cells above background levels and therefore was not chimeric. Accordingly, cells delivered by laser as described herein, and that may migrate to distant locations within the recipient body, remain functional and restore function to distant damaged organs. This experiment was repeated with syngeneic transplants in C57/BL6 mice as well.

### Example 10

### Fractional Laser can Attract Circulating Cells to a Laser Treated Site

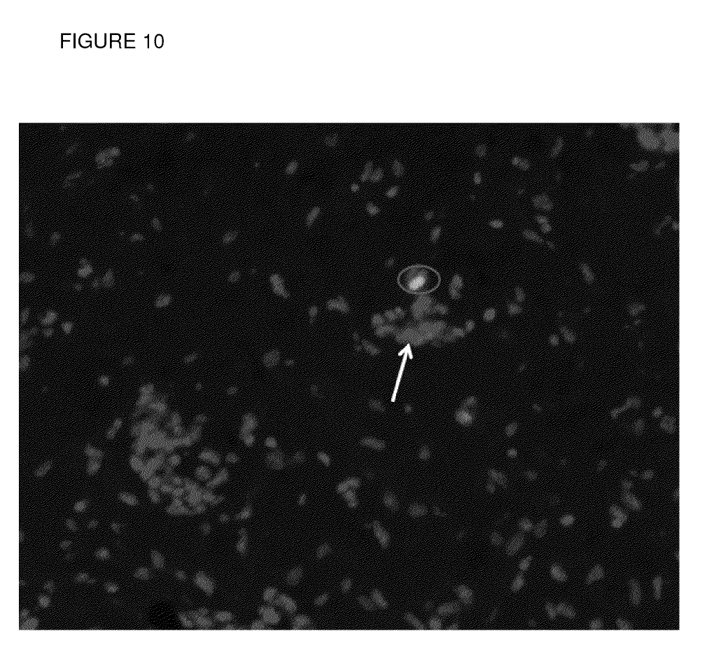

At a first site, skin tissue was wounded and treated with fractional laser ablation and labeled MSCs. At a second site far separated from the first site, skin tissue was wounded and treated with fractional laser and sterile saline, but not MSCs. After fourteen days, a tissue sample was obtained from the second site which had only been lasered. The slide was treated with DAPI to visualize nuclei, and analyzed by fluorescence microscopy as described above. As shown in FIG. 10, the arrow illustrates a blood vessel. The circle highlights a labeled MSC delivered by fractional laser at the first site, which has now migrated to the second site. Not only has the labeled MSC delivered by fractional laser entered the circulation, but it has also been attracted to the laser treated second site. Accordingly, laser treatment can be used to direct circulating cells to particular locations.

### Example 11

### Cells Delivered by Laser Remain Viable and are Capable of Division

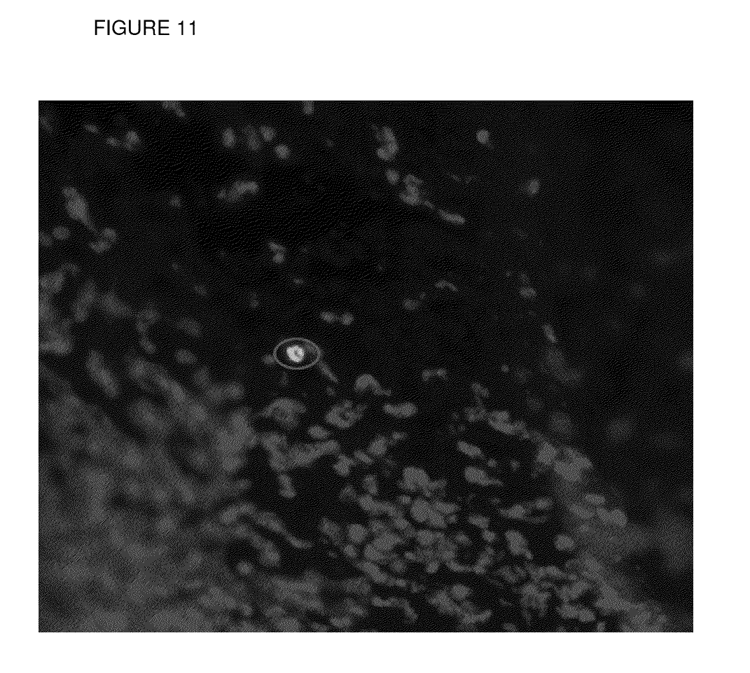

Skin tissue, epidermal and dermal, was wounded by the full thickness wound model as described above, then treated with laser ablation and transduced porcine MSC. Fourteen (14) days after treatment, a tissue sample including the crust formed over the wound was obtained, treated with DAPI to visualize nuclei, and analyzed by fluorescence microscopy as described above. As shown in FIG. 11, a labeled MSC (circled) in the crust displays chromosomal segregation in the nucleus, such as occurs during mitosis and cell division. Since the MSCs in the crust are capable of cell division, this indicates that not only are they functioning cells after migration, but also that they will likely secrete compounds and proteins to the surrounding crust and could serve as a delivery system to injured tissues.

### Example 12

### Cells Delivered by Laser can Facilitate Regrowth of Hair

C57/BL6 mice were pre-treated with ionizing whole-body radiation at doses sufficient to reduce and/or prevent hair regrowth. In this set of experiments, a dosage of 400 cGy gamma irradiation was administered using a Gammacell animal irradiator.

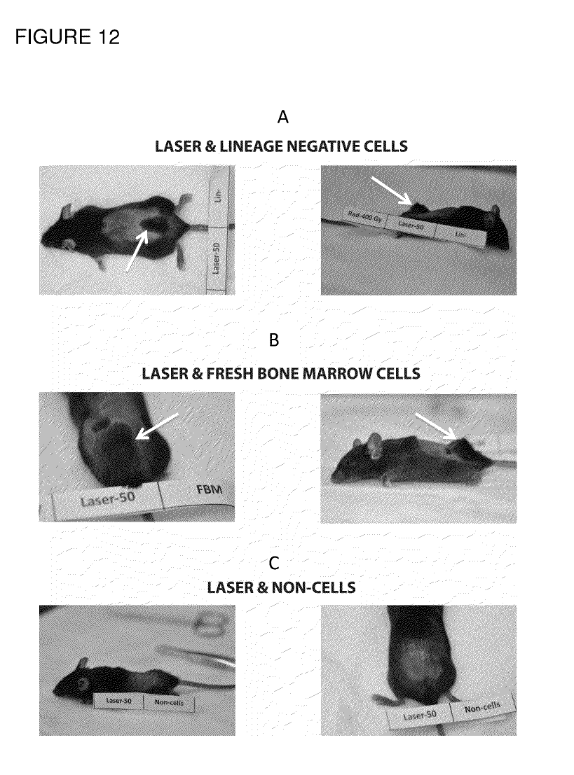

This is a biologically significant single radiation dose in this particular species. It would be expected to be sublethal but sufficient enough to produce both systemic and local effects including the inhibition of hair growth and pigmentation. Higher doses ranging from 800 to 1200 cGy (which are typically administered as half doses over two days to avoid acute toxicity) would more likely result in lethality due to gastrointestinal and/or bone marrow failure. A patch of hair on the back of each mouse was shaven, and a small area on the lower back of this shaven section was treated with fractional laser treatment. One million Lineage negative syngeneic bone marrow cells (lin(−)) cells (cells enriched in progenitor and stem cells and not expressing mature blood cell markers) were applied to a subset of test animals. Panel A of FIG. 12 shows that after four (4) weeks, these animals exhibited dramatic hair regrowth at the site of laser treatment and cell application, as shown by the patch of hair indicated by arrows. Not only did hair regrow in this area, but the regrown hair was black in color, rather than the grey color that is customarily seen in irradiated C57/BL6 mice when hair does regrow. Further, the hair regrowth resulting from laser and lin(−) cell treatment was denser in thickness and longer in length than even the surrounding original non-shaven hair. Another subset of test animals were subjected to ionizing radiation, shaven, laser treatment, and application of a suspension of one million syngeneic total bone marrow cells (meaning a mixed population of cells, some of which express mature blood cell markers and others not expressing these markers). As shown in Panel B of FIG. 12, after four (4) weeks these animals similarly exhibited hair regrowth at the treatment site, as indicated by arrows. This regrowth was also black in color, and denser and longer than surrounding original hair. Panel C of FIG. 12 shows the control group that was only treated with radiation and laser treatment, but no cells. As is readily apparent, these control animals showed little to no hair regrowth within the treated area after four (4) weeks. These results clearly indicate a biological effect in the induction of hair regrowth when cells are delivered locally to the skin using fractional laser delivery.

### Example 13

### Laser Assisted Delivery of Antibacterial Agent is Effective Against Biofilms

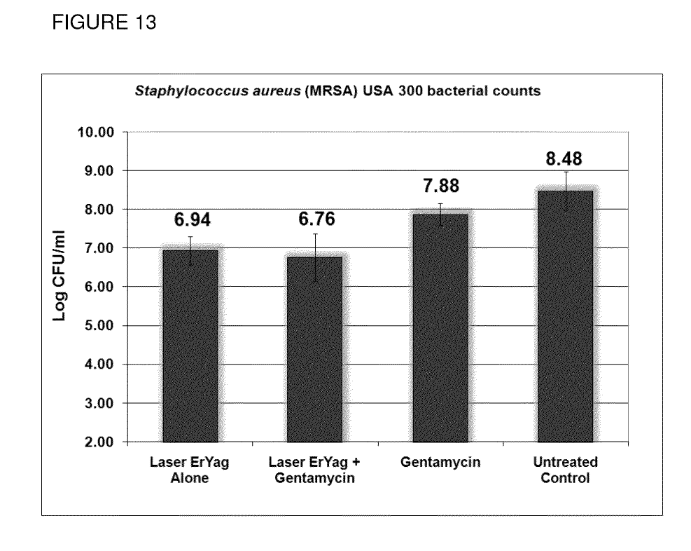

Methicillin resistant Staphylococcus aureus (MRSA) biofilms were created using a deep partial thickness wound model (described above). Wounds were randomly assigned to one of the following treatment groups: 1) untreated control, 2) ErYag, 3) ErYag plus gentamycin or 3) gentamycin alone. Twenty four hours after treatment, MRSA was recovered. As seen in FIG. 13, gentamycin alone and laser ErYag treatment alone were able to reduce MRSA counts in wounds by 0.6 and 1.5 Log CFU/ml, respectively. However in combination (Laser+gentamycin) were able to reduce MRSA counts by 1.72 Log CFU/ml. This decrease represents a 98.09% reduction in MRSA.

### Example 14

### Stem Cells Delivered by Fractional Laser can Induce the Release of Endogenous Stem Cells into the Circulation

Mesenchymal stem cells (MSCs) were labeled using a lentivirus as before, and approximately one million of these labeled cells were delivered by fractional laser per treatment area, which varied between 1 and 4 cm squared depending on the laser used—Er:YAG or C02—to the skin of a pig that had second degree burn wounds. Circulating non-labeled MSCs were then measured in equal volumes of blood (approximately 5 to 7 cc, obtained from a limb or ear vein using a vacutainer and butterfly needle) taken from treated pigs at days 5, 7, 14 and 21. The graph of circulating cells represents MSCs that were not delivered via laser but rather were MSCs released from endogenous sources (likely the bone marrow) of the treated animal.

The top line (square data points) of FIG. 14 represents circulating released MSC in an animal that received allogeneic (from another pig donor) bone marrow derived MSC to the skin using laser. The middle line (circular data points) of FIG. 14 represents circulating released MSC in an animal that received its own (autologous) labeled bone marrow derived MSC to the skin using laser. The bottom line (triangular data points) of FIG. 14, mostly observed as points along the x-axis, represents samples derived from an animal (with second degree burns) that did not receive MSCs to the skin but was treated with laser. No detectable cells were released. These results indicate that the present invention actually stimulates and/or enhances the subject's own endogenous stem cell production, including release of stem cells into circulation from tissues, such as bone marrow, and also a greater growth potential in stem cells once circulating. This is in striking contrast to known methods in which it has traditionally only been feasible to stimulate stem cell release by the administration of hematopoietic hormones. Moreover, only a relatively small treatment area and applied cell count is capable of inducing a large number of stem cells into circulation by release and subsequent robust growth. Approximately 1 million cells applied to laser treated areas stimulated many endogenous stem cells circulating within 5 days with an increase in circulating endogenous stem cells in 7 days. This increase in circulating endogenous stem cells also persisted for at least 3 weeks, which is not seen with other known stem cell therapies. This indicates that the present invention can be used to enhance and enrich a subject's own endogenous stem cells circulating in the blood. Accordingly, it may be used to stimulate such production to enable harvesting of stem cells from blood circulating in the patient. Since blood samples are far easier, less invasive, less painful, and less expensive than current stem cell harvesting methods of bone marrow extraction, the present invention provides still further benefits, which may be used to more easily harvest stem cells for other applications.

### Example 15

### Stem Cells Delivered with Fractional Laser can Alter the Immune Response Leading to a Better Clinical Outcome

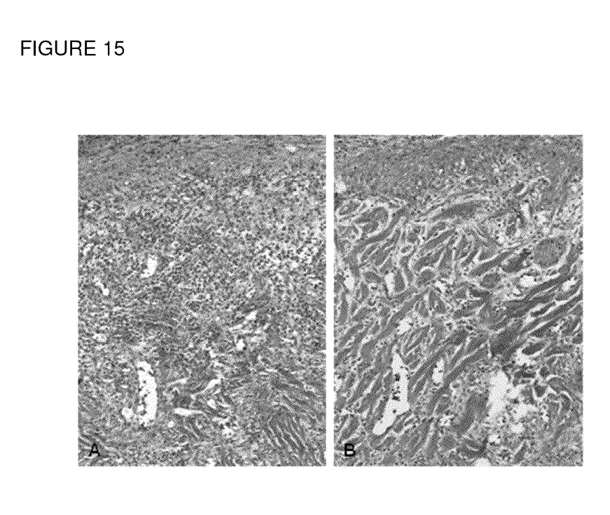

To test whether cells delivered by laser-assisted delivery can alter the immune response in burns in porcine skin, both epidermal and dermal, was injured by the burn wound protocol as described above, then treated with fractional laser ablation and subsequent application of YFP-transduced MSC or fractional laser alone. As seen in FIG. 15, panel (A), there is a robust inflammatory infiltrate noted at day 5 in the laser only treated burn wound. This infiltrate consists numerous polymorphonuclear leukocytes (PMNs). The inflammatory process associated with burns wounds is known to be a primary cause of morbidity associated with burn injuries, due to ischemia reperfusion injury largely mediated by PMNs and the production of reactive oxygen species. In burn wounds treated with fractional laser and MSCs, shown in FIG. 15, panel (B), the inflammatory response has been greatly attenuated with few PMNs present. This response is also associated with reduced scarring as has been illustrated in the above figures. This finding illustrates that delivery of stem cells with fractional laser can effectively produce a favorable immune response that will result in an improved clinical outcome.

### Example 16

### Stem Cells Delivered Systemically can be Attracted to Fractional Treated Sites to Produce a Therapeutic Outcome

C57/BL6 mice were pretreated with ionizing radiation as before, shaved, and given bone marrow stem cells by IV injection.

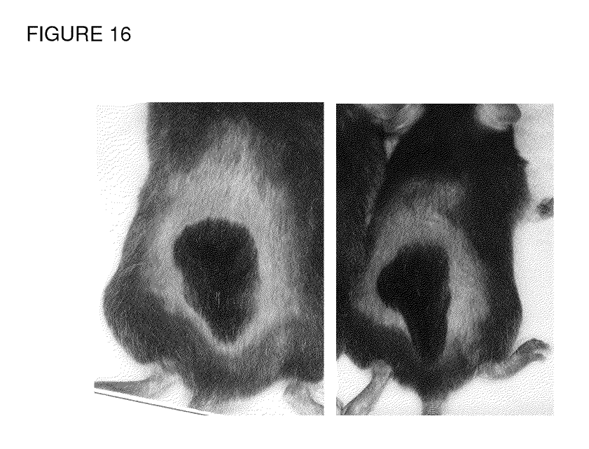

Subsequently, fractional laser treatment was applied in a square shape to a small area on the lower shaved back of each mouse. FIG. 16 shows dramatic hair re-growth in the area where the laser treatment was applied. The hair again was noted to be darker, longer and denser than adjacent areas that were not shaved. Mice treated with laser alone did not exhibit this hair growth. This experiment illustrates the ability of fractional laser to attract circulating cells (including stem cells) to the laser site and effect a beneficial change. It is also notable that the hair regrowth pattern does not follow precisely the pattern of laser application or stem cell application.

Together, the results presented in the Examples reveal that viable and functional bioactive agents (e.g. stem cells) can be readily delivered to tissues at an area of laser ablation, as well as systemically to tissues distal to the area of laser ablation. Furthermore, laser ablation can be used to direct circulating cells to a particular location, whether those cells were initially delivered by laser ablation or not. Laser delivery of bioactive agents, such as stem cells, also stimulates the production and/or release of endogenous stem cells in the subject, thus boosting therapeutic effects and repair. Laser ablation in concert with administration of bioactive agents further results in less inflammation, crusting, and local edema with reduced evidence of scarring in tissues. These results suggest that laser-assisted delivery of bioactive agents, as well as laser ablated explants serving as delivery matrices, are viable treatments for both localized and systemic disorders.

It is to be appreciated that the Detailed Description section, and not the Abstract section, is intended to be used to interpret the claims. The Abstract section may set forth one or more but not all exemplary embodiments of the present invention as contemplated by the inventor(s), and thus, are not intended to limit the present invention and the appended claims in any way.

The foregoing description of the specific embodiments should fully reveal the general nature of the invention so that others can, by applying knowledge within the skill of the art, readily modify and/or adapt for various applications such specific embodiments, without undue experimentation, without departing from the general concept of the present invention. Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents. Moreover, the breadth and scope of the present invention should not be limited by any of the above-described exemplary embodiments, but should similarly be defined only in accordance with the following claims and their equivalents.

### REFERENCES

1. Anderson R R, Parrish J A. Selective photothermolysis: precise microsurgery by selective absorption of pulsed radiation. Science. 1993, 220:524-527.
2. Manstein, D. D., Herron, G. S., Sink, R. K., Tanner, H., and Anderson, R. R. Fractional photothermolysis: a new concept for cutaneous remodeling using microscopic patterns of thermal injury. Lasers in Surgery and Medicine. 2004, 34:426-438.

1. A method of delivering one or more bioactive agents to a subject, comprising:
applying laser light to a target site on the subject, wherein the target site is spaced apart from a distal site to be affected by at least one bioactive agent,
creating at least one channel in the tissue of the target site with the laser light, and
administering one or more bioactive agent to the target site at said channel for migration into the channel and to the distal site.

2. The method as recited in claim 1 wherein creating at least one channel with laser light causes a reaction in the subject that promotes migration of the one or more bioactive agent.

3. The method as recited in claim 1 wherein applying laser light and creating at least one channel are sufficient to create an injury response at least 250 microns below the surface of the tissue of the target site.

4. The method as recited in claim 3 wherein the injury response is a laser-generated injury response.

5. The method as recited in claim 1 wherein creating at least one channel includes creating at least one channel having a predetermined depth of at least 300 microns as measured from a surface of the tissue at the target site.

6. The method as recited in claim 5 wherein creating at least one channel includes creating at least one channel having a predetermined depth in the range of about 300 to 2000 microns as measured from the surface of the tissue at the target site.

7. The method as recited in claim 1 wherein the tissue at the target site is skin.

8. The method as recited in claim 7 wherein creating at least one channel includes creating at least one channel having a depth at least sufficient to penetrate the dermal layer of skin.

9. The method as recited in claim 1 wherein applying a laser includes applying ablative laser light to the target site.

10. The method as recited in claim 9 wherein applying a laser includes applying fractional ablative laser light to the target site.

11. The method as recited in claim 1 wherein the at least one channel includes an opening at the surface of the tissue at the target site.

12. The method as recited in claim 11 wherein applying one or more bioactive agents includes applying one or more bioactive agents at the opening of the at least one channel.

13. The method as recited in claim 1 wherein the at least one channel is a coagulated channel.

14. The method as recited in claim 1 wherein the at least one channel comprises a width of about 400 microns or less.

15. The method as recited in claim 14 wherein the at least one channel comprises a width of about 200 microns or less.

16. The method as recited in claim 15 wherein the at least one channel comprises a width in the range of about 10 to 200 microns.

17. The method as recited in claim 1 further comprising creating a plurality of channels of predetermined depths in the tissue at the target site.

18. The method as recited in claim 17 further comprising creating a plurality of channels in the range of about 400 to 800 channels per centimeter square of target site.

19. The method as recited in claim 17 wherein each of the plurality of channels defines an opening in the tissue of the target site.

20. The method as recited in claim 19 wherein applying one or more bioactive agents includes applying one or more bioactive agents at the openings of the plurality of channels.

21. The method as recited in claim 1 wherein the at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

22. The method as recited in claim 1 wherein the one or more bioactive agents includes at least one type of functional cell.

23. The method as recited in claim 22 wherein administering includes administering approximately 1 million cells or less to the at least one channel at the target site.

24. The method as recited in claim 22 wherein the one or more bioactive agents includes at least one type of stem cell.

25. The method as recited in claim 24 wherein the one or more bioactive agents includes mesenchymal stem cells.

26. The method as recited in claim 22 wherein the one or more bioactive agents includes at least one type of adult cell.

27. The method as recited in claim 22 wherein the one or more bioactive agents includes at least lymphocytes.

28. The method as recited in claim 22 wherein the one or more bioactive agents includes at least one type of progenitor cell.

29. The method as recited in claim 22 wherein the one or more bioactive agents includes a mixture of functional cells.

30. The method as recited in claim 29 wherein the one or more bioactive agents includes a mixture of at least one type of stem cell and a second type of cell.

31. The method as recited in claim 30 wherein the second type of cell is progenitor cells.

32. The method as recited in claim 30 wherein the second type of cell is adult cells.

33. The method as recited in claim 30 wherein the stem cells are mesenchymal stem cells.

34. The method as recited in claim 30 wherein the at least one type of stem cell and the second type of cell have different potencies.

35. The method as recited in claim 1 further comprising applying laser light to the distal site, creating at least one channel at the distal site so as to direct the at least one bioactive agent to the distal site.

36. The method as recited in claim 1 wherein the at least one bioactive agent includes at least one corrective gene or corrective gene product capable of providing therapeutic benefit for a condition resulting from a corresponding aberrant gene or aberrant gene product.

37. The method as recited in claim 36 wherein the corrective gene product comprises RNA or protein.

38. The method as recited in claim 36 wherein the at least one bioactive agent comprises cells having at least one corrective gene or corrective gene product and capable of expressing the corrective gene or corrective gene product.

39. The method as recited in claim 36 wherein the at least one bioactive agent comprises a mixture of a first set of cells having at least one corrective gene or corrective gene product and capable of expressing said corrective gene or corrective gene product, and a second set of cells defined as at least one type of stem cell.

40. A method of delivering one or more bioactive agents to a subject, comprising:
applying laser light to a target site on the subject, wherein the target site is spaced apart from a distal site to be affected by at least one bioactive agent,
creating at least one channel of a predetermined depth in the tissue of the target site and defining an opening of the at least one channel at a surface of the target site tissue,
administering one or more bioactive agents at the opening of the at least one channel for migration into the channel and to the distal site, and
applying laser light to the distal site, thereby creating at least one channel at the distal site so as to direct the at least one bioactive agent to the distal site.

41. The method as recited in claim 40 wherein the predetermined depth of the at least one channel is at least 300 microns as measured from a surface of the tissue at the target site.

42. The method as recited in claim 41 wherein the predetermined depth of the at least one channel is in the range of about 300 to 2000 microns as measured from the surface of the tissue at the target site.

43. The method as recited in claim 40 wherein the tissue at the target site is skin.

44. The method as recited in claim 43 wherein creating at least one channel includes creating at least one channel having a depth at least sufficient to penetrate the dermal layer of skin.

45. The method as recited in claim 40 wherein the at least one channel comprises a width of about 400 microns or less.

46. The method as recited in claim 45 wherein the at least one channel comprises a width of about 200 microns or less.

47. The method as recited in claim 46 wherein the at least one channel comprises a width in the range of about 10 to 200 microns.

48. The method as recited in claim 40 wherein applying a laser includes applying ablative laser light to the target site.

49. The method as recited in claim 48 wherein the laser light is generated by a fractional ablative laser.

50. The method as recited in claim 40 wherein the at least one channel at the target site is coagulated.

51. The method as recited in claim 40 wherein the at least one channel at the distal site is coagulated.

52. The method as recited in claim 40 further comprising creating a plurality of channels in the tissue of the target site.

53. The method as recited in claim 52 further comprising creating a plurality of channels in the range of about 400 to 800 channels per centimeter square of target site.

54. The method as recited in claim 40 further comprising creating a plurality of channels in the tissue of the distal site.

55. The method as recited in claim 40 wherein the at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

56. The method as recited in claim 40 wherein the at least one bioactive agent includes at least one type of functional cell.

57. The method as recited in claim 56 wherein applying includes administering approximately 1 million cells or less to the at least one channel at the target site.

58. A method of directing at least one bioactive agent to a particular site within a subject, comprising:
identifying a particular site within a subject at which to direct at least one bioactive agent present in the subject,
applying laser light to the identified particular site, and
creating at least one channel with laser light at the identified site.

59. The method as recited in claim 58 wherein creating at least one channel with laser light causes a reaction in the subject that promotes migration of the one or more bioactive agent to the identified site.

60. The method as recited in claim 58 wherein applying laser light and creating at least one channel are sufficient to create an injury response at least 250 microns below the surface of the tissue at the identified site.

61. The method as recited in claim 60 wherein the injury response is a laser-generated injury response.

62. The method as recited in claim 58 wherein creating at least one channel includes creating at least one channel having a predetermined depth of at least 300 microns as measured from a surface of the identified site.

63. The method as recited in claim 62 wherein creating at least one channel includes creating at least one channel having a predetermined depth in the range of about 300 to 2000 microns as measured from the surface of the identified site.

64. The method as recited in claim 58 wherein the at least one bioactive agent is circulating in the subject.

65. The method as recited in claim 58 further comprising introducing the at least one bioactive agent into the subject.

66. The method as recited in claim 65 wherein introducing the at least one bioactive agent occurs prior to creating at least one channel with laser light at the identified site.

67. The method as recited in claim 65 wherein introducing the at least one bioactive agent occurs by injection.

68. The method as recited in claim 58 wherein the at least one bioactive agent includes at least one type of functional cell.

69. The method as recited in claim 58 wherein the at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

70. The method as recited in claim 58 further comprising creating a plurality of channels at the identified particular site with the laser light.

71. The method as recited in claim 58 wherein applying laser light includes applying ablative laser light.

72. The method as recited in claim 71 wherein applying laser light includes applying fractional ablative laser light.

73. A laser-assisted method of treating a systemic condition, comprising:
applying laser light to a target site of a subject, wherein the target site is spaced apart from a site affected by the condition to be treated,
creating at least one channel of a predetermined depth at the target site using the laser,
administering at least one bioactive agent to the target site of the subject at said channel for migration into said channel and systemic delivery to the affected site.

74. The method as recited in claim 73 wherein creating at least one channel with laser light causes a reaction in the subject that promotes systemic migration of the one or more bioactive agent.

75. The method as recited in claim 73 wherein applying laser light and creating at least one channel are sufficient to create an injury response at least 250 microns below the surface of the tissue of the target site.

76. The method as recited in claim 75 wherein the injury response is a laser-generated injury response.

77. The method as recited in claim 73 wherein the predetermined depth of the at least one channel is at least 300 microns as measured from a surface of the tissue at the target site.

78. The method as recited in claim 77 wherein the predetermined depth of the at least one channel is in the range of about 300 to 2000 microns as measured from the surface of the tissue at the target site.

79. The method as recited in claim 73 wherein the tissue at the target site is skin.

80. The method as recited in claim 79 wherein creating at least one channel includes creating at least one channel having a depth at least sufficient to penetrate the dermal layer of skin.

81. The method as recited in claim 73 wherein applying a laser includes applying ablative laser light to the target site.

82. The method as recited in claim 81 wherein applying a laser includes applying fractional ablative laser.

83. The method as recited in claim 73 wherein the condition to be treated is at least one of tissue damage, organ damage, myocardial infarction, chronic tissue disease, chronic lung disease, reduced immune function, reduced hematopoietic function, vascular disorders, artery disease, stroke, lymphedema, carcinoma, tumor, organ loss, partial organ loss, and tissue loss.

84. The method as recited in claim 73 wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

85. The method as recited in claim 73 wherein the at least one bioactive agent comprises at least one type of functional cells.

86. The method as recited in claim 73 wherein the at least one bioactive agent includes at least a vaccine for vaccination against a particular and predetermined antigen.

87. Use of at least one bioactive agent applied to an opening of at least one channel created by laser ablation for treating a systemic condition.

88. The use as recited in claim 87 wherein the systemic condition is at least one of tissue damage, organ damage, myocardial infarction, chronic tissue disease, chronic lung disease, reduced immune function, reduced hematopoietic function, vascular disorders, artery disease, stroke, lymphedema, carcinoma, tumor, organ loss, partial organ loss, and tissue loss.

89. The use as recited in claim 87 wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

90. The use as recited in claim 87 wherein said at least one bioactive agent comprises at least one type of functional cells.

91. The use as recited in claim 90 wherein said at least one bioactive agent comprises a mixture of cells.

92. The use as recited in claim 91 wherein said mixture of cells comprises a heterogenous mixture of cells.

93. The use as recited in claim 87 wherein said at least one bioactive agent comprises approximately 1 million cells or less administered to the at least one channel.

94. Use of at least one bioactive agent applied to an opening of at least one channel created by laser ablation for vaccination against a particular and predetermined antigen.

95. The use as recited in claim 94 wherein said at least one bioactive agent comprises a vaccine.

96. A method of stimulating endogenous stem cell production comprising:
identifying a target site on a subject,
applying laser light to the identified target site,
creating at least one channel at the identified target site with the laser light, and
administering at least one bioactive agent to the at least one channel, wherein the at least one bioactive agent includes at least one type of functional cell.

97. The method as recited in claim 96 wherein the at least one bioactive agent includes at least one type of stem cell.

98. The method as recited in claim 97 wherein the at least one type of bioactive agent includes mesenchymal stem cells.

99. The method as recited in claim 96 wherein administering includes administering approximately 1 million functional cells or less to the at least one channel at the target site.

100. The method as recited in claim 96 wherein the at least one type of functional cell applied to the channel is the same type of cell as the endogenous cells whose circulation is stimulated.

101. The method as recited in claim 96 wherein applying laser light and creating at least one channel are sufficient to create an injury response at least 250 microns below the surface of the tissue of the target site.

102. The method as recited in claim 101 wherein the injury response is a laser-generated injury response.

103. The method as recited in claim 96 wherein creating at least one channel includes creating at least one channel having a predetermined depth of at least 300 microns as measured from a surface of the tissue at the target site.

104. The method as recited in claim 103 wherein creating at least one channel includes creating at least one channel having a predetermined depth in the range of about 300 to 2000 microns as measured from the surface of the tissue at the target site.

105. The method as recited in claim 96 wherein the tissue at the target site is skin.

106. The method as recited in claim 105 wherein creating at least one channel includes creating at least one channel having a depth at least sufficient to penetrate the dermal layer of skin.

107. The method as recited in claim 96 wherein applying laser light includes applying ablative laser light to the identified target site.

108. The method as recited in claim 107 wherein applying laser light includes applying fractional ablative laser light to the identified target site.

109. The method as recited in claim 96 further comprising creating a plurality of channels at the identified target site with the laser light.

110. The method as recited in claim 109 wherein administering at least one bioactive agent comprises administering the at least one bioactive agent to the plurality of channels at the identified target site.

111. The method as recited in claim 96 wherein creating at least one channel at the identified target site with the laser light further comprises creating said at least one channel at the identified target site with the laser light to cause a laser-generated injury within said at least one channel at which said at least one bioactive agent is administered, said laser-generated injury in said at least one channel at which said at least one bioactive agent is administered stimulating the release of a greater number of endogenous stem cells into circulation in the subject than circulate under non-laser generated injury conditions.

112. The method as recited in claim 96 wherein creating at least one channel at the identified target site with the laser light further comprises creating said at least one channel at the identified target site with the laser light to cause a laser-generated injury within said at least one channel at which said at least one bioactive agent is administered, said laser-generated injury in said at least one channel at which said at least one bioactive agent is administered stimulating the growth of endogenous stem cells circulating in the subject than grow in circulation under non-laser generated injury conditions.

113. The method as recited in claim 96 further comprising harvesting endogenous stem cells from blood circulating in the subject.

114. A system for delivery of a bioactive agent, comprising:
a laser capable of producing laser light for creating at least one channel of a predetermined depth in a target tissue at a target site, thereby defining an opening at the surface of the target tissue, and
at least one bioactive agent disposable at the opening of the at least one channel and capable of migration into and through the at least one channel and systemic delivery to a distal site for beneficial effect at the distal site.

115. The system as recited in claim 114 wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

116. The system as recited in claim 114 wherein said at least one bioactive agent includes at least one type of functional cell.

117. The system as recited in claim 114 wherein said at least one bioactive agent includes about 1 million functional cells or less.

118. The system as recited in claim 114 wherein said at least one bioactive agent includes at least one type of stem cell.

119. The system as recited in claim 118 wherein said at least one bioactive agent includes mesenchymal stem cells.

120. The system as recited in claim 114 wherein said at least one bioactive agent comprises a mixture of cells.

121. The system as recited in claim 120 wherein said mixture of cells comprises a heterogenous mixture of cells.

122. The system as recited in claim 114 wherein said at least one channel is a coagulated channel.

123. The system as recited in claim 114 further comprising a plurality of channels of predetermined depths in said target tissue, wherein said plurality of channels collectively define a matrix.

124. The system as recited in claim 114 wherein said laser is an ablative laser.

125. The system as recited in claim 124 wherein said laser is a fractional ablative laser.

126. The system as recited in claim 114 wherein said laser is capable of creating channels having a predetermined depth of at least 300 microns.

127. The system as recited in claim 126 wherein said laser is capable of creating channels having a predetermined depth in the range of about 300 to 2000 microns.

128. The system as recited in claim 114 wherein said laser is capable of creating an injury response at least 250 microns below the surface of the tissue of the target site.

129. The system as recited in claim 128 wherein the injury response is a laser-generated injury response.

130. The system as recited in claim 114 wherein said laser is capable of creating at least one channel having a width of about 400 microns or less.

131. The system as recited in claim 130 wherein said laser is capable of creating at least one channel having a width of about 200 microns or less.

132. The system as recited in claim 131 wherein said laser is capable of creating at least one channel having a width in the range of about 10 to 200 microns.

133. The system as recited in claim 114 wherein said laser is capable of creating a plurality of channels of predetermined depths in the tissue at the target site.

134. The system as recited in claim 133 wherein said laser is capable of creating a plurality of channels in the range of about 400 to 800 channels per centimeter square of target site.

135. A kit for the delivery of at least one bioactive agent, comprising:
at least one bioactive agent, and
instructions for application of said at least one bioactive agent to at least one laser-created channel in a target tissue for systemic delivery of said bioactive agent to a distal site spaced apart from the target tissue.

136. The kit as recited in claim 135 wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

137. The kit as recited in claim 135 wherein said at least one bioactive agent comprises at least one type of functioning cells.

138. The kit as recited in claim 137 wherein said at least one bioactive agent includes at least one type of stem cell.

139. The kit as recited in claim 137 wherein said at least one bioactive agent comprises a mixture of a first type of functioning cell and at least a second type of functioning cell.

140. The kit as recited in claim 135 further comprising a laser capable of producing laser light to create said at least one channel.

141. The kit as recited in claim 140 wherein said laser comprises an ablative laser.

142. The kit as recited in claim 140 wherein said laser comprises a fractional ablative laser.

143. The kit as recited in claim 140 wherein said laser is mobile.

144. The kit as recited in claim 140 wherein said laser is hand-held.

145. The kit as recited in claim 135 further comprising a tissue explant.

146. The kit as recited in claim 145 further comprising instructions for application of said at least one bioactive agent to said tissue explant to form a seeded explant and implantation of said seeded tissue explant within a subject.

147. A method of delivering a bioactive agent to a subject, comprising:
applying laser light to a tissue explant,
creating at least one channel of a predetermined depth in the tissue explant with the laser light,
administering at least one bioactive agent to the tissue explant to obtain a seeded tissue explant, and
implanting the seeded tissue explant into a subject.

148. The method as recited in claim 147 wherein the at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

149. The method as recited in claim 147 wherein the at least one bioactive agent comprises at least one type of functional cells.

150. The method as recited in claim 149 wherein the at least one bioactive agent comprises at least one type of stem cell.

151. The method as recited in claim 147 wherein implanting comprises implanting the seeded tissue explant into a tissue of the subject.

152. The method as recited in claim 147 wherein implanting comprises implanting the seeded tissue explant into an organ of the subject.

153. The method as recited in claim 147 wherein implanting comprises implanting the seeded explant at an implantation site to a predetermined depth within the subject so as to enable systemic movement of the at least one bioactive agent from the seeded explant to sites within the subject distant from the implantation site.

154. The method as recited in claim 147 wherein implanting occurs at a surface of the subject.

155. The method as recited in claim 147 wherein implanting occurs subcutaneously.

156. The method as recited in claim 147 wherein applying laser light includes applying ablative laser light.

157. The method as recited in claim 156 wherein applying laser light includes applying fractional ablative laser light.

158. The method as recited in claim 147 further comprising creating a plurality of channels in the tissue explant.

159. The method as recited in claim 147 wherein the at least one bioactive agent includes at least one corrective gene or corrective gene product capable of providing therapeutic benefit for a condition resulting from a corresponding aberrant gene or aberrant gene product.

160. The method as recited in claim 159 wherein the corrective gene product comprises RNA or protein.

161. The method as recited in claim 159 wherein the at least one bioactive agent comprises cells having at least one corrective gene or corrective gene product and capable of expressing the corrective gene or corrective gene product.

162. A system for delivering bioactive agent to tissue, comprising:
a tissue explant,
at least one channel disposed throughout said tissue explants and having an opening defined at a surface of said tissue explant, and
at least one bioactive agent applied to said opening of said at least one channel.

163. The system as recited in claim 162 wherein said tissue explant is of biologic origin.

164. The system as recited in claim 162 wherein said tissue explant is synthetic.

165. The system as recited in claim 162 wherein said at least one bioactive agent includes one of cells, protein, peptide, peptide fragment, nucleic acid, nucleotide fragment, gene, pharmaceutical compound, therapeutic compound, medicament, small molecule, aptamer, and combinations thereof.

166. The system as recited in claim 162 wherein said at least one bioactive agent comprises at least one type of functional cells.

167. The system as recited in claim 166 wherein said at least one bioactive agent comprises at least one type of stem cell.

168. The system as recited in claim 162 further comprising a plurality of channels disposed throughout said tissue explant so as to define a matrix of channels.

169. The system as recited in claim 162 further comprising a laser capable of producing laser light to create said at least one channel in said tissue explant.

170. The system as recited in claim 162 wherein said laser is an ablative laser.

171. The system as recited in claim 170 wherein said laser is a fractional ablative laser.

172. Use of a tissue explant including at least one laser ablated channel and at least one bioactive agent applied to said at least one laser ablated channel for treating a systemic condition.

173. A method of treating a biofilm on a subject comprising applying laser light to an affected site on the subject sufficient to disrupt the biofilm.

174. The method as recited in claim 173 further comprising administering at least one bioactive agent to the affected site.

175. The method as recited in claim 174 wherein the at least one bioactive agent comprises one of cells, protein, peptide, peptide fragment, nucleic acid, nucleotide fragment, gene, pharmaceutical compound, therapeutic compound, medicament, small molecule, aptamer, and combinations thereof.

176. The method as recited in claim 175 wherein the at least one bioactive agent is an antimicrobial agent.

177. The method as recited in claim 175 wherein the at least one bioactive agent is at least one type of functional cell capable of expressing an antimicrobial agent.

178. The method of claim 174 further comprising applying a subsequent round of laser treatment to the affected site.
